(19) United States
(12) Patent Application Publication
(10) Pub. No.: US 2025/0003973 A1
(43) Pub. Date: Jan. 2, 2025

(54) SYNTHETIC CONSTRUCTS AND METHODS FOR PROCESSING ANTIGEN-SPECIFIC CELLS

(71) Applicants: Fred Hutchinson Cancer Center, Seattle, WA (US); ImmunoScape Pte. Ltd., Singapore (SG)

(72) Inventors: Evan Newell, Seattle, WA (US); Zilei Liu, San Diego, CA (US)

(73) Assignees: Fred Hutchinson Cancer Center, Seattle, WA (US); ImmunoScape Pte. Ltd., Singapore (SG)

(21) Appl. No.: 18/711,441

(22) PCT Filed: Dec. 7, 2022
(86) PCT No.: PCT/US2022/081087

§ 371 Date: May 17, 2024

Related U.S. Application Data
(60) Provisional application No. 63/286,723, filed on Dec. 7, 2021.

(51) Int. Cl. G01N 33/58; C07K 16/28; C12Q 1/6806; G01N 33/68

(52) U.S. Cl. CPC G01N 33/582; C07K 16/2803; C07K 16/2809; C12Q 1/6806; G01N 33/6848

(57) ABSTRACT

The present disclosure provides methods for detecting antigen-specific cells, such as T cells or B cells, using a proximity ligation assay (PLA) in which at least one of the PLA probes is a peptide-major histocompatibility (MHC) multimer or a B cell specific antigen bound to an oligonucleotide. Also provided are kits for use in such methods.

### CROSS-REFERENCE TO RELATED APPLICATION

This application claims the benefit of U.S. Provisional Application No. 63/286,723, filed Dec. 7, 2021, the disclosure of which is hereby incorporated by reference in its entirety.

### STATEMENT REGARDING SEQUENCE LISTING

The Sequence Listing XML associated with this application is provided in XML format and is hereby incorporated by reference into the specification. The name of the XML file containing the sequence listing is 1896-P47WO2_Seq_List_20221205. The XML file is 24 KB; was created on Dec. 5, 2022; and is being submitted via Patent Center with the filing of the specification.

### BACKGROUND

Peptide-major histocompatibility (MHC) tetramer staining allows direct detection of antigen specific cells in a sample. Identification of rare antigen-specific T cells can be achieved by staining cells with peptide-MHC tetramers that are loaded with various peptides. However, non-specific staining can hamper the ability to detect very rare cells. Further, the signal to noise ratio can vary significantly, which is also a significant limitation in trying to detect very rare cells.

There is therefore a need in the art for improved methods to detect antigen specific cells that are more specific and that produce a higher signal-to-noise ratio. The present disclosure fulfills this need and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

According to certain of the herein described embodiments, there is provided synthetic constructs and a method for processing (e.g., detecting, separating, isolating, enriching, or purifying) target cell(s) in a sample, comprising: contacting the target cell with a cell selector component for which the target cell is specific, the cell selector component being bound to a first oligonucleotide; contacting the target cell with a targeting component specific for a second antigen on the target cell, the targeting component being bound to a second oligonucleotide; forming a nucleic acid template by ligating the first oligonucleotide and the second oligonucleotide; and amplifying the nucleic acid template. In specific aspects, provided is a method for detecting the target cell.

In certain other embodiments there is provided a kit for processing (e.g., detecting, separating, isolating, enriching, or purifying) a target cell in a sample comprising: a cell selector component for which the target cell is specific, the cell selector component being bound to a first oligonucleotide; a targeting component specific for a second antigen on the target cell, the targeting component being bound to a second oligonucleotide; a ligation reagent; and written instructions for using the cell selector component and the targeting component to detect the target cell.

In additional embodiments, provided herein are synthetic constructs comprising a cell selector component; a first oligonucleotide bound to the peptide-MHC multimer; a second oligonucleotide bound to the first oligonucleotide; and a targeting component bound to the second oligonucleotide. As used herein, a “synthetic construct” refers to an artificially constructed structure that comprises a combination of peptides and oligonucleotides bound together.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1. In this embodiment, a target T cell is contacted with a peptide-MHC multimer loaded with a peptide antigen for which the TCR is specific. Each monomer of the peptide-MHC multimer comprises a myc-tag, a his-tag, or a fluorescent tag, and the monomers are multimerized with streptavidin. A secondary anti-myc, anti-his, or anti-fluorochrome antibody bound to an oligonucleotide contacts the targeting component and binds to the myc-tag, to the his-tag, or to the fluorescent tag. The T cell is also contacted with an anti-CD3 or an anti-TCR antibody, or an antibody against an associated molecule, where the antibody is bound to a second oligonucleotide. Proximity ligation assay (PLA) detection oligonucleotides are then amplified to create PLA products, which are labeled, e.g., a fluorescent label, a heavy metal label, an oligonucleotide label, etc.

FIG. 2. In this embodiment, a target T cell is contacted with a peptide-MHC multimer loaded with a peptide antigen for which the TCR is specific. The monomers are multimerized with streptavidin, and an oligonucleotide is bound to a monomer or the streptavidin. The T cell is also contacted with an anti-TCR or anti-CD3 Fab that is bound to a second oligonucleotide. Proximity ligation assay (PLA) detection oligonucleotides are then amplified to create PLA products, which are labeled, e.g., a fluorescent label, a heavy metal label, an oligonucleotide tag, etc.

FIG. 3. In this embodiment, a target B cell is contacted with an antigen of the B cell receptor (BCR). The antigen is bound to an oligonucleotide. The B cell is also contacted with an anti-BCR or anti-CD79 antibody, or an antibody against an associated molecule, where the antibody is bound to a second oligonucleotide. Proximity ligation assay (PLA) detection oligonucleotides are then amplified to create PLA products, which are labeled, e.g., a fluorescent label, a heavy metal label, an oligonucleotide tag, etc.

FIGS. 4A and 4B. FIG. 4(A) Workflow for performing proximity ligation assay (PLA) targeting CD3 and TCR using human PBMCs based on secondary antibody-probes. In brief, human PBMCs were incubated with rabbit anti-hCD3 (clone SP7) and mouse anti-hTCRα/β-BV510 (clone IP26) antibodies. After fixation in 2% PFA and blocking, the cells were incubated with Duolink® anti-rIgG-PLUS and Duolink® anti-mIgG-MINUS. Then the cells were subjected to ligation, amplification, and detection steps using Duolink® flowPLA kit according to manual. FIG. 4(B) Flow cytometric analysis showing TCRα/β+ cells exhibit specific PLA signal.

FIGS. 5A and 5B. FIG. 5(A) Demonstration of performing PLA targeting CD3 and TCR using human PBMCs based on primary antibody-probes. In brief, Duolink® PLUS and MINUS oligo probes were introduced on mouse anti-hCD3 (clone OKT3) and mouse anti-hTCRα/D (clone IP26) antibodies respectively using Duolink® probe maker kit according to user manual. Then human PBMCs were fixed in 2% PFA followed by incubating with anti-hCD3-PLUS and anti-hTCRα/β-MINUS. Then the cells were subjected to ligation, amplification, and detection steps using Duolink® flowPLA kit according to user manual. FIG. 5(B) Flow cytometric analysis showing a distinct PLA+ population (left panel) which mainly consisted of CD4+ and CD8+ cells (right panel). P-Values were calculated using t-test. Error bars represent the mean±SEM (n=4); ns: P≥0.05; *P≤0.1; **P≤0.01; ***P≤0.001; ****P≤0.0001.

FIGS. 6A and 6B. FIG. 6(A) Demonstration of performing PLA targeting CD3 and TCR using human PBMCs with anti-hCD3-PLUS and anti-PE-MINUS. In brief, Duolink® PLUS and MINUS oligo probes were introduced on mouse anti-hCD3 (clone OKT3) and rabbit anti-PE (polyclonal) antibodies respectively using Duolink® probe maker kit according to user manual. Then human PBMCs were incubated with anti-TCRα/β-PE. After fixation in 2% PFA and blocking, the cells were incubated with anti-hCD3-PLUS and anti-PE-MINUS antibodies and subsequently subjected to ligation, amplification, and detection steps according to Duolink® flowPLA kit user manual. In a control group, no anti-hCD3-PLUS or anti-PE-MINUS antibodies were added. FIG. 6(B) Flow cytometric analysis and column chart showing TCRα/β+ population exhibits specific PLA signal (middle panel). P-Values were calculated using t-test. Error bars represent the mean±SEM (n=4); ns: P≥0.05; *P<0.1; **P<0.01; ***P<0.001; ****P<0.0001.

FIGS. 7A through 7C. FIG. 7(A) Demonstration of increasing the specificity of antigen-specific tetramer staining based on PLA. In brief, Duolink® PLUS and MINUS oligo probes were introduced on mouse anti-hCD3 (clone OKT3) and rabbit anti-PE (polyclonal) antibodies respectively using Duolink® probe maker kit according to user manual. Then human PBMCs were incubated with a PE-labelled p:MHCI tetramer bearing antigen CMV IE1 (VLEETSVML) (SEQ ID NO: 1). After fixation in 2% PFA and blocking, the cells were incubated with anti-hCD3-PLUS and anti-PE-MINUS antibodies and subsequently subjected to ligation, amplification, and detection steps according to Duolink® flowPLA kit user manual. In a control group, no anti-hCD3-PLUS or anti-PE-MINUS antibodies were added. FIG. 7(B) Flow cytometric analysis and column chart showing CD8+Tetramer+ antigen-specific population (antigen specific CD8 T cells) exhibits specific PLA signal (middle panel). FIG. 7(C) Flow cytometric analysis showing CD8+Tetramer+ population (light gray) exhibits significantly higher PLA signal than CD8-Tetramer+ population (dark gray). P-Values were calculated using t-test. Error bars represent the mean±SEM (n=3); ns: P≥0.05; *P≤0.1; **P≤0.01; ***P≤0.001; ****P≤0.0001.

FIGS. 8A and 8B. FIG. 8(A) Demonstration of increasing the specificity of antigen-specific tetramer staining based on Unfold PLA. In brief, human PBMCs were incubated with a peptide-MHCI tetramer-PE bearing antigen CMV IE1 (VLEETSVML) (SEQ ID NO: 1) or MART-1 (ELAGIGILTV) (SEQ ID NO: 2). After fixation in 2% PFA and blocking, the cells were incubated with mouse anti-hCD3 and rabbit anti-PE, followed by the incubation with secondary antibody-probes provided in the Unfold PLA kit. Subsequently the cells were subjected to ligation, amplification, and detection steps according to Unfold PLA kit user manual. CD8− population was used as a control to gate PLA+ and PLA− populations. FIG. 8(B) Flow cytometric analysis showing CD8+ Tetramer+ cells consisting of PLA+ and PLA− populations.

FIGS. 9A and 9B. FIG. 9(A) Experimental protocol for PLA using smart-tube fixed PBMCs. FIG. 9(B) Flow cytometric analysis showing the Smart-tube fixed PBMCs is compatible with PLA. PBMCs from donor HC08 were stained with the tetramer bearing CMV pp65 peptide and then fixed/preserved using commercial Smart-tubes. After thawing, the Smart-tube fixed PBMCs could be directly subjected to PLA procedure to achieve a strong and specific PLA signal.

FIGS. 10A through 10C. FIG. 10(A) Demonstration of the experiment for comparing the sensitivities and specificities between tetramer staining and PLA. In brief, CD8+ T cells transduced with MAGE-A1 or MAGE-A4-specific TCRs were pre-stained with CFSE and mixed with PBMCs from healthy donor SDBB020, which contain no MAGE-A1 or MAGE-A4-specific T cells at the cell percentage of 0.05%. Then tetramer staining and Unfold PLA were performed to evaluate corresponding sensitivity and specificity. FIG. 10(B) Flow cytometric analysis showing the sensitivity and specificity of detection of MAGE-A4 specific T cells using tetramer staining and/or PLA. FIG. 10(C) Flow cytometric analysis showing the sensitivity and specificity of detection of MAGE-A1 specific T cells using tetramer staining and/or PLA.

FIG. 11. Flow cytometric analysis showing the identification of EBV LMP2 and hTERT-specific CD8 T cells in PBMCs from donor SDBB132: in comparison of the tetramer staining of EBV LMP2 tetramer, hTERT (a TAA) tetramer showed high staining background, which led the difficulties of gating tetramer+ cells in CD8 population. PLA could help to identify TAA-specific T cells from the high staining background.

FIGS. 12A through 12D. FIG. 12(A) Demonstration of the detection of antigen-specific T cells with mass cytometry based on tetramer staining in combination with PLA. Donor: HC08; antigen: CMV pp65 (HLA A02). In brief, PBMCs were stained with a cocktail of tetramer-PE, tetramer-154Sm and tetramer-165Ho. Then Unfold PLA was performed using detection oligo-155Gd. Antigen-specific T cells could be determined by the signal of 154Sm, 165Ho and 155Gd. FIG. 12(B) Experimental protocol of unfold PLA for mass cytometry. FIG. 12(C) Gating strategy of mass cytometry analysis. FIG. 12(D) Mass cytometry analysis showing that double tetramer-metal (154Sm and 165Ho) positive CD8+ cells consist of 70% PLA-155Gd+ cells and 30% PLA-155Gd− cells, suggesting that 154Sm+/165Ho+/PLA-155Gd+CD8 cells were true CMV pp65-specific T cells. Cells not stained with anti-CD3/anti-PE antibodies were used as control.

FIGS. 13A and 13B. FIG. 13(A) Demonstration of the detection of antigen-specific T cells with mass cytometry based on tetramer staining in combination with PLA. Donor: SDBB035; antigen: MAGE-A1 (HLA A24). In brief, PBMCs were stained with a cocktail of tetramer-PE, tetramer-154Sm and tetramer-152Sm. Then Unfold PLA was performed using detection oligo-155Gd. Antigen-specific T cells could be determined by the signal of 154Sm, 152Sm and 155Gd. FIG. 13(B) Mass cytometry analysis showing the identification of tumor antigen-specific T cells using PLA in combination of tetramer barcoding strategy.

FIGS. 14A and 14B. FIG. 14(A) Demonstration of the detection of antigen-specific T cells with mass cytometry based on his-tag-tetramer staining in combination with PLA. Donor: SDBB059; antigen: CMV pp65 (HLA A02). In brief, tetramer was prepared using streptavidin-PE and MHC-I monomers bearing a his-tag. In this design, PLA signal could be generated using two primary antibody combinations: anti-CD3/anti-PE (left picture) and anti-CD3/anti-his tag (right picture). FIG. 14(B) Flow cytometry analysis showing unfold PLA signal using two primary antibody combinations: anti-CD3/anti-his tag (right panel) showed comparable PLA signal with anti-CD3/anti-PE (middle panel). Cells stained with no primary antibodies (anti-CD3, anti-PE or anti-his tag) were used as the negative control.

FIGS. 15A through 15C. FIG. 15(A) Demonstration of the detection of antigen-specific T cells with mass cytometry based on his tag-tetramer staining in combination with unfold PLA. Donor: SDBB059; antigen: CMV pp65 (HLA A02). In brief, tetramer was prepared using streptavidin-metals and MHC-I monomers bearing a his-tag. In this design, PLA signal could be generated using anti-anti-CD3/anti-anti-his tag. FIG. 15(B) Experimental protocol of unfold PLA for mass cytometry. FIG. 15(C) Flow cytometry analysis showing unfold PLA in combination of triple tetramer metal barcoding. In brief, 58.3% of 154Sm+173Yb+CD8 T cells also had 165Ho+ PLA+ signal, suggesting that this population consisted of high confidence CMV pp65 specific T cells. In contrast, the CD4+ population only showed background signal on PLA and tetramer-metal channels.

FIGS. 16A through 16C. Demonstration of PLA-based detection of antigen-specific T cells using probes directly conjugated to primary antibodies. Oligos were directly conjugated to primary antibodies (anti-CD3 and anti-PE). PBMCs from donor HC08 were stained with tetramer bearing CMV pp65 peptide, and PLA signal was generated with probes conjugated with primary versus secondary antibodies. FIG. 16(A) and FIG. 16(B) Flow cytometric analysis to compare the PLA signals produced by the combination of secondary antibody-probes (A) versus primary antibody-probes (B). FIG. 16(C) Statistical analysis of PLA signal/background ratios: In the CD8+ cell population, the method using primary antibody-probes results in significantly stronger signal to background ratio than the method using secondary antibody-probes. P-Values were calculated using t-test. Error bars represent the mean±SEM (n=3); ns: P≥0.05; *P≤0.1; **P≤0.01; ***P≤0.001; ****P≤0.0001.

FIG. 17. Flow cytometric analysis demonstrating that unfold PLA could be used to identify antigen-specific B cell in combination with antigen staining. The figure in middle-left panel illustrates the experimental design. In brief, PBMCs were stained with a cocktail of PE-labelled and PE/vio770-labelled SARS COVID-2 spike antigen complexes. Then mouse anti-CD79a/b was used together with rabbit anti-PE for the generation of PLA signal. Antigen+ dead cells were used as control for gating PLA.

FIGS. 18A through 18D. FIG. 18(A) Demonstration of the detection of antigen-specific T cells with single cell sequencing based on tetramer staining in combination with PLA. Donor: HC06; antigen: CMV pp65 (HLA B07). PBMCs were stained with tetramer-PE and oligo conjugated surface hashtags in two separate experiments. Unfold PLA was performed using detection flow cytometry. PLA+ antigen-specific T cells could be determined by the signal of far-red and sorted out using flow cytometry. The sorted Tetramer+ PLA+ cells from experiment one (hashtag6) and Tetramer− cells from experiment two (hashtag7) were mixed and prepared for single cell sequencing analysis using 10× Genomics. FIG. 18(B) Experimental workflow of unfold PLA for single cell sequencing. FIG. 18(C) Gating strategy of Tetramer− cells and Tetramer+ PLA+ cells for sorting. FIG. 18(D) Single cell sequencing analysis of TCR detected using 10× genomics single cell sequencing method. The Tetramer+ PLA+ cells and Tetramer− cells as shown in (C) are labelled with hashtag6 and hashtag7, respectively. Each dot represents a cell with TCR sequences detected and assigned to Tetramer+ PLA+ or Tetramer-PLA− groups according to their hashtags.

### DETAILED DESCRIPTION

In certain aspects, the present disclosure provides modified proximity ligation assays (PLA) for processing (e.g., detecting, separating, isolating, enriching, or purifying) antigen-specific cells in a sample using a cell selector component comprising a peptide-MHC multimer and a B cell specific antigen. Related synthetic constructs and kits are also described. The methods, synthetic constructs, and kits described herein can be used to identify rare antigen-specific cells (e.g., antigen-specific T cells) with increased specificity over traditional peptide-MHC tetramer staining processes. Advantageously, the methods, synthetic constructs, and kits described herein provide for reduced false positives by eliminating non-specific binding.

Prior to setting forth this disclosure in more detail, it can be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The term “consisting essentially of” limits the scope of a claim to the specified materials or steps, or to those that do not materially affect the basic characteristics of a claimed invention. For example, a protein domain, region, or module (e.g., a binding domain, hinge region, linker module) or a protein (which can have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 14%, 13%, 12%, 11%, 10%, 9%, 8%, 7%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

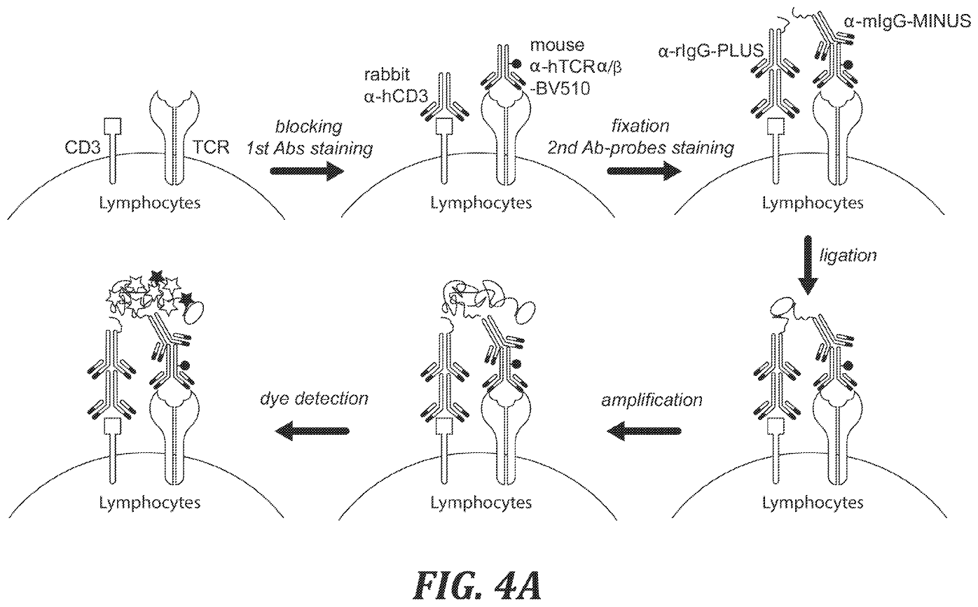

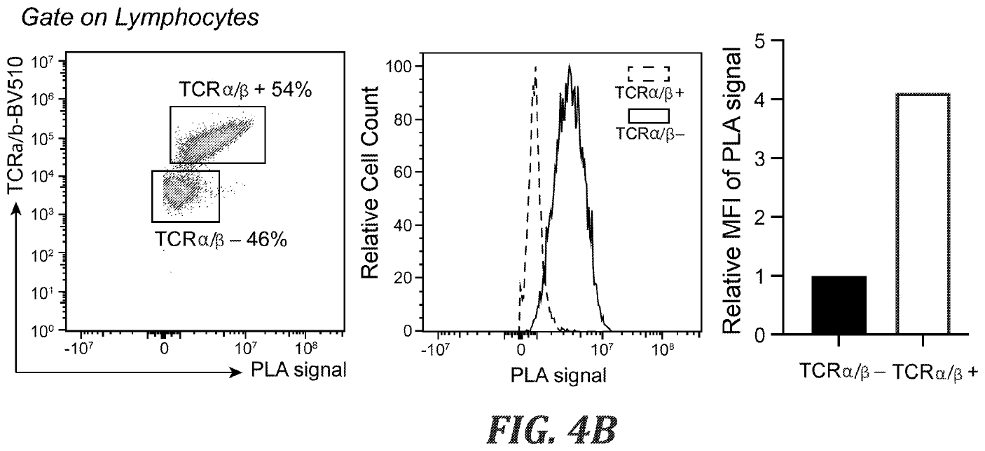

As used herein, “Proximity ligation assay” (PLA) is a technology used for detection of protein-protein interactions in situ (at distances <40 nm) at endogenous protein levels. This technology relies on the initial staining of two primary antibodies from different species to contiguous proteins of interest. Cells are then stained with corresponding secondary antibodies conjugated with PLA oligonucleotide probes (a PLUS and a MINUS probe). If the proteins of interest are close to each other, the DNA probes hybridize to make circular DNA. This DNA can be amplified and visualized by fluorescently-labeled, metal-labeled, or DNA-barcode-labeled complementary oligonucleotide probes as shown in FIG. 4A.

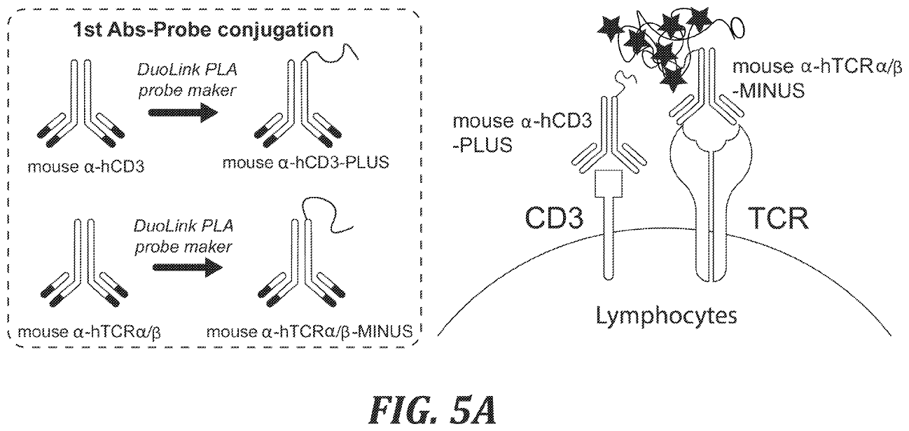

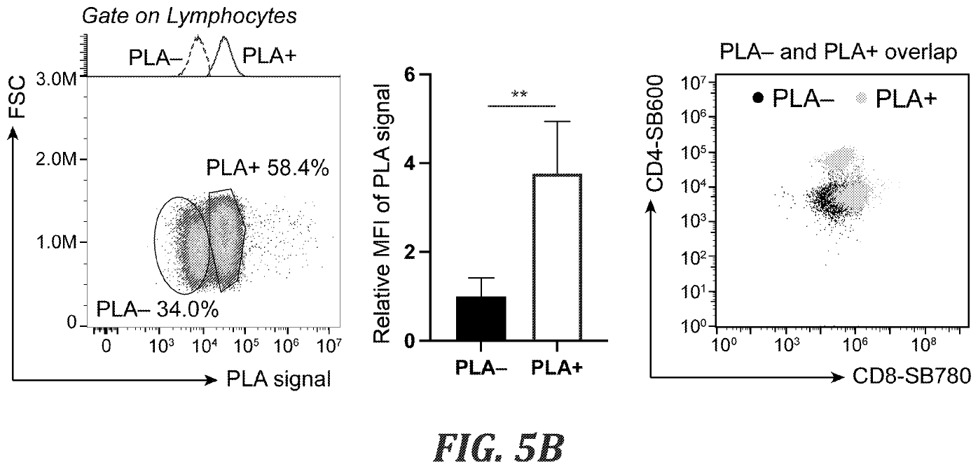

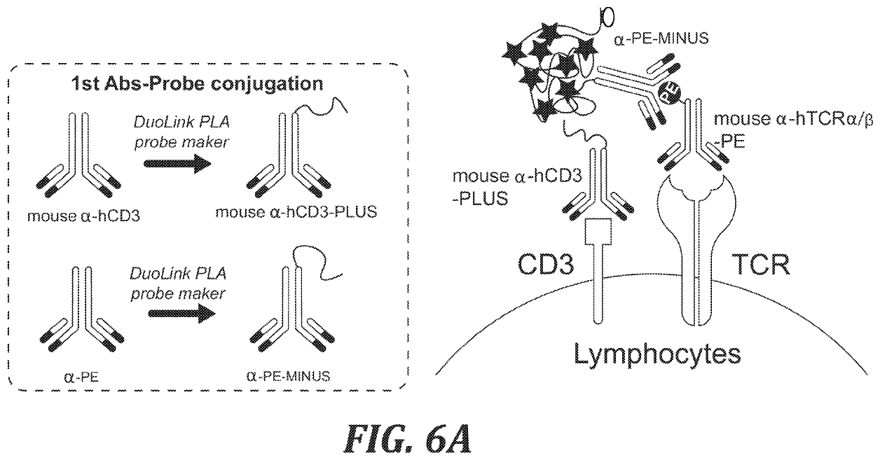

In an alternative approach, primary antibodies could be conjugated with PLA probes using Duolink® in situ PLA probe maker kits, and PLA could be performed without secondary antibodies (FIG. 5A). Since it was introduced in 2002, PLA has been widely used for the studies of signal protein, protein-protein interaction, and post-translational modifications with high sensitivity and specificity.

In another approach, multimer staining assay using peptide:MHC (p:MHC) multimers has been proven as a highly efficient approach for the detection of antigen-specific T cells in academic and industrial settings. In particular, a p:MHC tetramer can be made up of four biotinylated major histocompatibility complex (MHC) molecules bearing peptide antigens and one streptavidin in the center. A p:MHC multimer can be also made up of multiple biotinylated major histocompatibility complex (MHC) molecules bearing peptide antigens binding to a dextran-streptavidin scaffold in the center. Normally streptavidin or dextran is conjugated with a tag (e.g., fluorophore, metal, or oligonucleotide tag). If a T cell receptor (TCR) matches the peptide being presented by a MHC molecule, the antigen-specific T cell can be stained with the multimer and can be identified by flow cytometry, mass cytometry, or with next-generation DNA sequencing. Although multimer staining typically shows high specificity for the detection of antigen-specific T cells, unspecific tetramer staining can sometimes be observed, and it might be caused by the interactions other than TCR-peptide-MHC interaction.

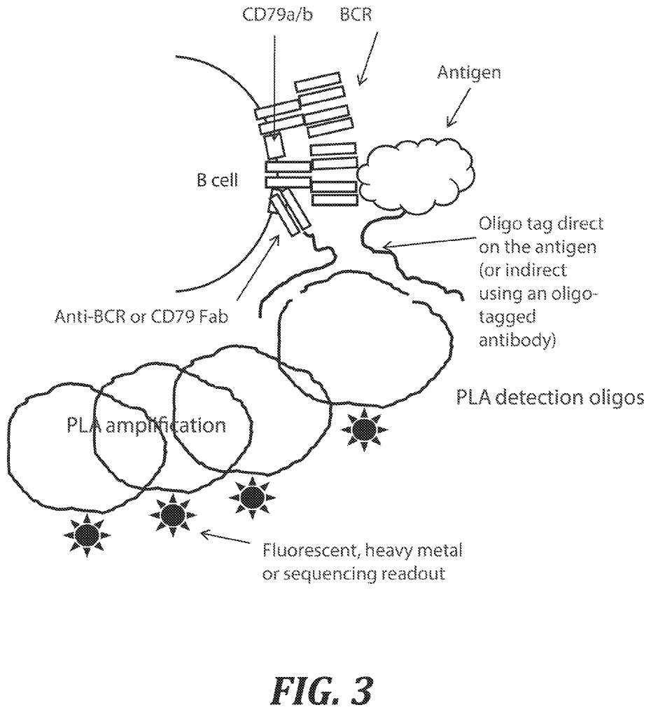

In still another approach, specific to B cells, the p:MHC tetramer is replaced by an antigen that B cells can recognize via their B cell receptor (BCR). In some embodiments, the antigen is specific to a particular B cell of interest. If a B cell binding to the B cell specific antigen has a corresponding cell surface target, e.g., cell surface receptor or cell surface antigen, specific to the targeting component, and the targets bound to the B cell specific antigen and the targeting component are close to each other, the DNA probes hybridize to make circular DNA. This DNA can be amplified and visualized by fluorescently-labeled, metal-labeled, or DNA-barcode-labeled complementary oligonucleotide probes as shown in FIG. 3.

For example, in some embodiments, a PLA assay can be performed using the following methods. The method can comprise assaying donor PBMC samples to assess frequencies of CMV-specific T cells. Next, live PBMCs are stained with myc-tagged, his-tagged, or fluorescent-tagged, or directly oligonucleotide-tagged peptide-MHC tetramers.

For example, a CMV-peptide loaded HLA-A0201 p:MHC tetramer can be used to stain the live PBMCs. The cells are then washed and fixed with paraformaldehyde. When myc-tagged, his-tagged, or fluorescent-tagged peptide-MHC tetramers are used the cells are then incubated with anti-CD3 and anti-myc, anti-his, or anti-fluorochrome antibodies conjugated with oligonucleotide PLA probes.

After washing, a ligase and a ligation buffer (e.g., Duolink® Ligation buffer) is added. The sample is washed again, an amplification buffer and polymerase are added, and the sample is incubated. The samples are then washed twice. The sample is then analyzed in a mass cytometer, flow cytometer or a fluorescence or confocal microscope, using at least a 20× objective.

As a control, a sample is run in parallel in which the peptide-MHC tetramer is not added. Alternatively, a sample can be run in parallel in which a negative control tetramer (i.e., a tetramer that would not be expected to bind any of the T cells). “Major histocompatibility complex molecules” (MHC molecules) refer to glycoproteins that deliver peptide antigens to a cell surface. MHC class I molecules are heterodimers consisting of a membrane spanning a chain (with three a domains) and a non-covalently associated 32 microglobulin. MHC class II molecules are composed of two transmembrane glycoproteins, a and R, both of which span the membrane. Each chain has two domains. MHC class I molecules deliver peptides originating in the cytosol to the cell surface, where peptide:MHC complex is recognized by CD8+ T cells. MHC class II molecules deliver peptides originating in the vesicular system to the cell surface, where they are recognized by CD4+ T cells. An MHC molecule can be from various animal species, including human, mouse, rat, or other mammals.

A “T cell” or “T lymphocyte” is an immune system cell that matures in the thymus and produces T cell receptors (TCRs), which can be obtained (enriched or isolated) from, for example, peripheral blood mononuclear cells (PBMCs). The term “T cell” refers to cells that show at least one phenotypic characteristic of a T cell or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage can be progenitor or precursor cells committed to the T cell lineage; CD25⁺ immature and inactivated T cells; cells that have undergone CD4 or CD8 linage commitment; thymocyte progenitor cells that are CD4⁺CD8⁺ double positive; single positive CD4⁺ or CD8⁺; TCRαβ or TCR γδ; or mature and functional or activated T cells. The term “T cells” encompasses naïve T cells (CD45 RA+, CCR7+, CD62L+, CD27+, CD45RO−), central memory T cells (CD45RO⁺, CD62L⁺, CD8⁺), effector memory T cells (CD45RA+, CD45RO−, CCR7−, CD62L−, CD27−), mucosal-associated invariant T cells, natural killer T cells, and tissue resident T cells.

After isolation of T cells, both cytotoxic (CD8+) and helper (CD4+) T cells can be sorted into naïve, memory, and effector T cell subpopulations, either before or after expansion. T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (T_{EM}, decreased expression of CD62L, CCR7, CD28, CD45RA, and increased expression of CD127 as compared to naïve T cells or T_{CM}). Effector T cells (T_{E}) refers to antigen-experienced CD8+ cytotoxic T lymphocytes that have decreased expression of CD62L, CCR7, CD28, and are positive for granzyme and perform as compared to T_{CM}. Helper T cells (T_{H}) are CD4+ cells that influence the activity of other immune cells by releasing cytokines. CD4+ T cells can activate and suppress an adaptive immune response, and which action is induced will depend on presence of other cells and signals. T cells can be collected in accordance with known techniques, and the various subpopulations or combinations thereof can be enriched or depleted by known techniques, such as by affinity binding to antibodies, flow cytometry, or immunomagnetic selection, or by techniques described herein. Other exemplary T cells include regulatory T cells, such as CD4+CD25+(Foxp3+) regulatory T cells and Treg17 cells, as well as Tr1, Th3, CD8+CD28+, and Qa-1 restricted T cells. “T cell receptor” (TCR) refers to a molecule found on the surface of T cells that, in association with CD3, is generally responsible for recognizing antigens bound to major histocompatibility complex (MHC) molecules. The TCR has a disulfide-linked heterodimer of the highly variable α and β chains (also known as TCRα and TCRβ, respectively) in most T cells. In a small subset of T cells, the TCR is made up of a heterodimer of variable γ and δ chains (also known as TCRγ and TCRδ, respectively). Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see Janeway et al., Immunobiology: The Immune System in Health and Disease, 3^{rd} Ed., Current Biology Publications, p. 4:33, 1997).

As used herein, “TCR complex” refers to a complex formed by the association of CD3 with TCR. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCRδ chain.

A “component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

The term “B cell” refers to cells that show at least one phenotypic characteristic of a B cell or a precursor or progenitor thereof that distinguishes the cells from other lymphoid cells, and cells of the erythroid or myeloid lineages. Such phenotypic characteristics can include expression of one or more proteins specific for B cells (e.g., CD19⁺, CD72+, CD24+, CD20⁺), or a physiological, morphological, functional, or immunological feature specific for a B cell. For example, cells of the B cell lineage can be progenitor or precursor cells committed to the B cell lineage (e.g., pre-pro-B cells, pro-B cells, and pre-B cells); immature and inactivated B cells or mature and functional or activated B cells. Thus, “B cells” encompass naïve B cells, plasma cells, regulatory B cells, marginal zone B cells, follicular B cells, lymphoplasmacytoid cells, plasmablast cells, and memory B cells (e.g., CD27⁺, IgD⁻).

“B cell receptor” (BCR) refers to a molecule found on the surface of B cells that, in association with CD79, controls the activation of B cells. The BCR is a membrane bound immunoglobulin molecule (e.g., IgD, IgM, IgA, IgG, or IgE) with an integral transmembrane region and an N-terminal immunoglobulin variable domain. In some embodiments, the BCR binds to an antigen as part of the cell selector process.

As used herein, “BCR complex” refers to a complex formed by the association of CD79 with BCR.

A “component of a BCR complex,” as used herein, refers to a BCR chain, a CD79 chain (i.e., CD79a or CD79b), or a complex formed by two or more BCR chains or CD79 chains.

Terms understood by those in the art of antibody technology are each given the meaning acquired in the art, unless expressly defined differently herein. The term “antibody” is used in the broadest sense and includes polyclonal and monoclonal antibodies. An “antibody” can refer to an intact antibody comprising at least two heavy (H) chains and two light (L) chains inter-connected by disulfide bonds, as well as an antigen-binding portion (or antigen-binding domain) of an intact antibody that has or retains the capacity to bind a target molecule. An antibody can be naturally occurring, recombinantly produced, genetically engineered, or modified forms of immunoglobulins, for example intrabodies, peptibodies, nanobodies, single domain antibodies, SMIPs, multispecific antibodies (e.g., bispecific antibodies, diabodies, triabodies, tetrabodies, tandem di-scFV, tandem tri-scFv, ADAPTIR). A monoclonal antibody or antigen-binding portion thereof can be non-human, chimeric, humanized, or human, preferably humanized or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988). “Antigen-binding portion” or “antigen-binding domain” of an intact antibody is meant to encompass an “antibody fragment,” which indicates a portion of an intact antibody and refers to the antigenic determining variable regions or complementary determining regions of an intact antibody. Examples of antibody fragments include, but are not limited to, Fab, Fab′, F(ab′)2, and Fv fragments, Fab′-SH, F(ab′)₂, diabodies, linear antibodies, scFv antibodies, VH, and multispecific antibodies formed from antibody fragments. A “Fab” (fragment antigen binding) is a portion of an antibody that binds to antigens and includes the variable region and CHI of the heavy chain linked to the light chain via an inter-chain disulfide bond. An antibody can be of any class or subclass, including IgG and subclasses thereof (IgG₁, IgG₂, IgG₃, IgG₄), IgM, IgE, IgA, and IgD.

The term “variable region” or “variable domain” of an antibody refers to the domain of an antibody heavy or light chain that is involved in binding of the antibody to antigen. The variable domains of the heavy chain and light chain (VH and VL, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single VH or VL domain can be sufficient to confer antigen-binding specificity. Furthermore, antibodies that bind a particular antigen can be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Antigen” or “Ag” as used herein refers to an immunogenic molecule that provokes an immune response. This immune response can involve antibody production, activation of specific immunologically-competent cells (e.g., T cells), or both. An antigen (immunogenic molecule) can be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen. In some embodiments, an antigen, i.e., B cell specific antigen, can be selected to bind to a specific B cell. In still other embodiments, the B cell specific antigen binds to the BCR as part of the cell selector process. In other embodiments, one of ordinary skill in the art, using standard techniques can select an antigen to bind to a particular B cell.

The term “epitope” or “antigenic epitope” includes any molecule, structure, amino acid sequence or protein determinant that is recognized and specifically bound by a cognate binding molecule, such as a T cell receptor (TCR) or other binding molecule, domain, or protein. Epitopic determinants generally contain chemically active surface groupings of molecules, such as amino acids or sugar side chains, and can have specific three-dimensional structural characteristics, as well as specific charge characteristics.

A “binding domain” (also referred to as a “binding region” or “binding moiety”), as used herein, refers to a molecule or portion thereof (e.g., peptide, such as an oligopeptide) that possesses the ability to specifically and non-covalently associate, unite, or combine with a target (e.g., a peptide-MHC complex). A binding domain includes any naturally occurring, synthetic, semi-synthetic, or recombinantly produced binding partner for a biological molecule, a molecular complex (i.e., complex comprising two or more biological molecules), or other target of interest. Exemplary binding domains include single chain immunoglobulin variable regions (e.g., scTCR, scFv), receptor ectodomains, ligands (e.g., cytokines, chemokines), or synthetic polypeptides selected for their specific ability to bind to a biological molecule, a molecular complex or other target of interest.

As used herein, “specifically binds” or “specific for” refers to an association or union of a binding protein (e.g., an antibody) or a binding domain (or fusion protein thereof) to a target molecule (e.g., an antigen) with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding proteins or binding domains (or fusion proteins thereof) can be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding proteins or binding domains refer to those binding proteins or binding domains having a Kₐ of up to 10⁷ M⁻¹, up to 10⁶ M⁻¹, up to 10⁵ M⁻¹. Alternatively, affinity can be defined as an equilibrium dissociation constant (Kₐ) of a particular binding interaction with units of M (e.g., 10⁻⁵ M to 10⁻¹³ M).

In certain embodiments, a receptor or binding domain can have “enhanced affinity,” which refers to selected or engineered receptors or binding domains with stronger binding to a target antigen than a wild type (or parent) binding domain. For example, enhanced affinity can be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof.

A variety of assays are known for identifying binding domains of the present disclosure that specifically bind a particular target, as well as determining binding domain or binding protein affinities, such as Western blot, ELISA, analytical ultracentrifugation, spectroscopy, surface plasmon resonance (Biacore®) analysis, MHC tetramer assay (see, e.g., Scatchard et al., Ann. N. Y. Acad. Sci. 51:660, 1949; Wilson, Science 295:2103, 2002; Wolff et al., Cancer Res. 53:2560, 1993; Altman et al., Science 274:94-96, 1996; and U.S. Pat. Nos. 5,283,173, 5,468,614, or the equivalent).

Principles of antigen processing by antigen presenting cells (APC) (such as dendritic cells, macrophages, lymphocytes or other cell types), and of antigen presentation by APC to T cells, including major histocompatibility complex (MHC)-restricted presentation between immunocompatible (e.g., sharing at least one allelic form of an MHC gene that is relevant for antigen presentation) APC and T cells, are well established (see, e.g., Murphy, Janeway's Immunobiology (8^{th} Ed.) 2011 Garland Science, NY; chapters 6, 9 and 16). For example, processed antigen peptides originating in the cytosol (e.g., tumor antigen, intracellular pathogen) are generally from about 7 amino acids to about 11 amino acids in length and will associate with class I MHC molecules, whereas peptides processed in the vesicular system (e.g., bacterial, viral) will vary in length from about 10 amino acids to about 25 amino acids and associate with class II MHC molecules.

As used herein, the terms “peptide” refers to a compound comprised of amino acid residues covalently linked by peptide bonds. A peptide must contain at least two amino acids, and no limitation is placed on the maximum number of amino acids that can comprise a peptide's sequence. As used herein, the term refers to both short chains, which also commonly are referred to in the art as peptides, oligopeptides, and oligomers, for example, and to longer chains, which generally are referred to in the art as proteins, of which there are many types. “Peptides” include, for example, biologically active fragments, substantially homologous peptides, oligopeptides, homodimers, heterodimers, variants of peptides, modified peptides, derivatives, analogs, fusion proteins, among others. The peptides include natural peptides, recombinant peptides, synthetic peptides, or a combination thereof.

As used herein, “nucleic acid” or “nucleic acid molecule” refers to deoxyribonucleic acid (DNA), ribonucleic acid (RNA), or a combination thereof. For example, polynucleotides (e.g., oligonucleotides), including those generated by the polymerase chain reaction (PCR) or by in vitro transcription, and to those generated by any of ligation, scission, endonuclease action, or exonuclease action. In certain embodiments, the nucleic acids of the present disclosure are produced by PCR. Nucleic acids can be composed of monomers that are naturally occurring nucleotides (such as deoxyribonucleotides and ribonucleotides), analogs of naturally occurring nucleotides (e.g., anti-enantiomeric forms of naturally occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. In various embodiments, modified internucleotide linkages are used. Modified internucleotide linkages are well known in the art and include methylphosphonates, phosphorothioates, phosphorodithionates, phosphoroamidites and phosphate ester linkages. Nucleic acid molecules can be either single stranded or double stranded.

“Oligonucleotide” as used herein refers to a single stranded nucleic acid molecule. An oligonucleotide is not necessarily physically derived from any existing or natural sequence, and can be generated in any manner, including chemical synthesis, DNA replication, reverse transcription, or a combination thereof. In embodiments, an oligonucleotide is comprised of no more than 50 nucleotides.

The term “hybridization” as used herein refers to any process by which a first strand of nucleic acid binds with a second strand of nucleic acid through base pairing. Hybridization can occur between fully complementary nucleic acid strands or between “substantially complementary” nucleic acid strands that contain minor regions of mismatch.

As used herein, a probe is a polynucleotide that is “specific,” for a target sequence if, when used under sufficiently stringent conditions, the probe hybridizes primarily only to the target nucleic acid. Typically, a probe is specific for a target sequence if the probe-target duplex stability is greater than the stability of a duplex formed between the probe and any other sequence found in the sample. One of skill in the art will recognize that various factors, such as salt conditions as well as base composition of the probe and the location of the mismatches, will affect the specificity of the probe, and that routine experimental confirmation of the probe specificity will be needed in most cases. Hybridization conditions can be chosen under which the probe can form stable duplexes only with a target sequence. Thus, the use of target-specific probes under suitably stringent conditions enables the specific amplification of those target sequences which contain the target probe binding sites. The use of sequence-specific conditions enables the specific binding of the probes to the target sequences which contain the exactly complementary probe binding sites.

Conditions under which only fully complementary nucleic acid strands will hybridize are referred to as “stringent hybridization conditions” or “sequence-specific hybridization conditions”. The term “stringent” as used herein refers to hybridization conditions that are commonly understood in the art to define the conditions of the hybridization procedure. Stringency conditions can be low, high, or medium, as those terms are commonly known in the art and well recognized by one of ordinary skill. In various embodiments, stringent conditions can include, for example, highly stringent conditions, and/or moderately stringent (i.e., medium stringency) conditions.

Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art.

As used herein, “complementary” refers to a nucleic acid molecule that can form hydrogen bond(s) with another nucleic acid molecule by either traditional Watson-Crick base pairing or other non-traditional types of pairing (e.g., Hoogsteen or reversed Hoogsteen hydrogen bonding) between complementary nucleosides or nucleotides.

It is understood in the art that a nucleic acid molecule need not be 100% complementary to a target nucleotide sequence to be specifically hybridizable. That is two or more nucleic acid molecules can be less than fully complementary and is indicated by a percentage of contiguous residues in a nucleic acid molecule that can form hydrogen bonds with a second nucleic acid molecule. For example, if a first nucleic acid molecule has 10 nucleotides and a second nucleic acid molecule has 10 nucleotides, then base pairing 5 of 5, 6, 7, 8, 9, or 10 nucleotides between the first and second nucleic acid molecules represents 50%, 60%, 70%, 80%, 90%, and 100% complementarity, respectively. “Perfectly” or “fully” complementary nucleic acid molecules means those in which all the contiguous residues of a first nucleic acid molecule will hydrogen bond with the same number of contiguous residues in a second nucleic acid molecule, wherein the nucleic acid molecules either both have the same number of nucleotides (i.e., have the same length) or the two molecules have different lengths.

The term “hybridization complex” as used herein refers to a complex formed between two nucleotide sequences by virtue of the formation of hydrogen bonds between complementary G and C bases and between complementary A and T bases; these hydrogen bonds can be further stabilized by base stacking interactions. The two complementary nucleotide sequences hydrogen bond in an antiparallel configuration. A hybridization complex can be formed in solution (e.g., Cot or Rot analysis) or between one nucleotide sequence present in solution and another nucleotide sequence immobilized on a solid support (e.g., paper, membranes, filters, chips, pins or glass slides, or any other appropriate substrate to which cells and/or nucleic acids have been fixed).

“Optional” or “optionally” means that the subsequently described event or circumstances can or cannot occur, and that the description includes instances where said event or circumstance occurs and instances in which it does not.

“Fluorescent” refers to a molecule which is capable of absorbing light of a particular frequency and emitting light of a different frequency. Fluorescence is well-known to those of ordinary skill in the art.

“Contacting” is used in accordance with its plain ordinary meaning and refers to the process of allowing at least two distinct species (e.g. nucleic acids and/or proteins) to become sufficiently proximal to react, interact, or physically touch. It should be appreciated, that the resulting reaction product can be produced directly from a reaction between the added reagents or from an intermediate from one or more of the added reagents which can be produced in the reaction mixture.

“Forming” is used in accordance with its plain ordinary meaning and refers to the process of producing a structure (e.g., a nucleic acid template) by coupling two or more components or otherwise performing a reaction to alter the structure of at least one of the components.

As noted above, described herein are synthetic constructs and methods comprising a proximity ligation assay (PLA) that can be used to process (e.g., detect, separate, isolate, enrich, or purify) antigen-specific cells. As used herein, PLA refers to a process for processing (e.g., detecting, separating, isolating, enriching, or purifying) a cell of interest on which an antigen is located in close proximity to a receptor (e.g., no more than 50 nanometers (nm)), which is referred to herein as a “target cell.” Such processes can include contacting a T cell in a sample with (1) a peptide-MHC multimer that is loaded with a peptide antigen for which the T cell is specific, and (2) a targeting component that is specific for a receptor on the T cell. In other embodiments, such process can include contacting a B cell target in a sample with (1) an antigen for which the B cell is specific, and (2) a targeting component that is specific for a receptor on the B cell. The antigen responsible for binding to a certain B cell target is herein referred to as a “B cell specific antigen.” The peptide-MHC multimer and the B cell specific antigen are herein referred to as the “cell selector component” of the synthetic construct. Additionally, the component specific for a receptor on a T cell and the component specific for a receptor on a B cell are referred to as the “targeting component” of the synthetic construct.

The process for contacting the T cell with the peptide-MHC multimer is often referred to as “peptide-MHC multimer staining.” MHC-peptide tetramer staining methods for detecting antigen specific T cells are well known in the art (e.g., Altman et al, 1996, Science 274:94-96; Kalergis et al., 2000, J. Immunol. Methods 234:61-70; Xu and Screaton, 2002, J. Immunol. Methods 268:21-8; James et al., J. Vis. Exp. 25:1167).

A “peptide-MHC multimer” refers to a structure comprising at least two MHC molecule monomers, where at least one of the monomers is loaded with a peptide. Any suitable peptide-MHC multimer can be used. For example, a dimer, a tetramer, a dodecamer, or a dextramer. In embodiments, the peptide-MHC multimer is a peptide-MHC tetramer. In certain embodiments, the MHC-peptide multimer comprises MHC Class I molecules. In other embodiments, the MHC-peptide multimer comprises MHC Class II molecules.

As is understood, an MHC molecule is “loaded with” a peptide when the peptide is bound in the peptide-binding cleft, between the α₁ and α₂ domains for an MHC Class I molecule and between the α₁ and β₁ domains for the MHC Class II molecule. The peptide-MHC multimers can be loaded with any suitable peptide for which an antigen-specific cell is specific. In other words, the peptide loaded is at least part of the epitope to which the antigen specific cell binds. Any suitable peptide antigens can be used, and the amino acid sequence of such peptide antigens can be determined using any suitable methods. Examples of epitope sequences are described, e.g., in Newell, et al., Nature Biotechnology, 2013, 31(7), 623-629. In some embodiments, each MHC molecule comprises an identical peptide having an amino acid sequence that is cognate (e.g., identical or related to) at least one antigen, wherein the multimer is capable of binding the target cell (e.g., a T cell).

In various embodiments, each MHC molecule has a mass of about 50 kilodalton (kDa). In embodiments, the binding affinity between a monomer and the TCR is on the order of 10 micromolar. In some embodiments, the binding affinity between a monomer and the TCR is at least 100 micromolar.

In particular embodiments, the peptide-MHC multimer comprises streptavidin (e.g., a recombinant streptavidin). Each of the MHC molecules can be tagged with a biotin molecule. In embodiments, the biotinylated MHC/peptides are tetramerized by the addition of streptavidin. In embodiments, the streptavidin is bound to a dextran backbone, and the biotinylated MHC/peptides are multimerized by the addition of streptavidin-dextran backbone. In some such embodiments, the streptavidin or the dextran is labeled with a fluorescent, metal, or DNA barcode tag. Such multimers can be detected by flow cytometry, mass cytometry, or sequencing via the tag. Examples of such peptide-MHC tetramers and the related staining processes are described, for example, in Davis, et al, Nature Reviews Immunology, 2011, 11(8), 551-558, and Bentzen, et al, Nature Biotechnology, 2016, 34(10), 1037-1045, which are incorporated by reference herein for their teachings regarding the same. In specific embodiments, the peptide-MHC multimer is a tetramer constructed using recombinant streptavidin and site-specifically biotinylated MHC. In particular embodiments, the peptide-MHC multimer is a dextramer that comprises streptavidin bound to poly-dextran. In other embodiments, the peptide-MHC multimer is a dimer or pentameter.

In various embodiments, the targeting component comprises a binding domain that is specific for a receptor on the target cell. As used herein, “receptor” refers to a peptide (e.g., at the surface of a target cell) that receives a signal. For example, a component of a TCR complex (for example, CD3 or the TCR) or a component of a BCR complex (for example, CD79, such as CD79a or CD79b, or the BCR). A binding domain suitable for use in the present disclosure can be any antigen-binding polypeptide that provides a stable bond between the peptide and the antigen. In embodiments, a suitable binding domain will generally have a binding affinity in the micromolar to sub-nanomolar range. A binding domain can comprise a natural antibody, synthetic or recombinant antibody construct, or a binding fragment thereof. For example, a binding domain can comprise a full-length heavy chain, Fab fragment, Fab′, F(ab′)2, variable heavy chain domain (VH domain), variable light chain domain (VL domain), domain antibody (dAb), single domain camelid antibody (VHH), complementary determining region (CDR), or single chain antibody fragment (scFv). Other examples of binding domains include single chain T cell receptors (scTCRs), extracellular domains of receptors, ligands for cell surface receptors/molecules, tumor binding proteins/peptides, and cytokines. In some embodiments, a binding domain is murine, chimeric, human, or humanized. In some embodiments, the targeting component comprises an antibody or antigen binding fragment thereof, a fusion protein, an aptamer, a small molecule specific for an antigen, variable lymphocyte receptors (VLR, as described in, e.g., Velikovsky, et al., Nat. Struct. Mol. Biol. 2009; 16(7):725-30), or a combination thereof.

In particular embodiments, the targeting component comprises an antibody. In certain embodiments, the targeting component is an anti-TCR antibody. In some such embodiments, the anti-TCR antibody is specific for CD3. In other such embodiments, the anti-TCR antibody is specific for another TCR associated peptide. In certain other embodiments, the targeting component comprises a Fab fragment.

In embodiments, the targeting component comprises a peptide-MHC multimer that is loaded with a peptide antigen for which the target cell is specific. Any suitable peptide antigen can be used, and the amino acid sequence of such peptide antigens can be determined using any suitable methods. Examples of epitope sequences are described, e.g., in Newell, et al., Nature Biotechnology, 2013, 31(7), 623-629.

In specific embodiments, the peptide-MHC multimer is a peptide-MHC tetramer and the targeting component is an anti-TCR antibody. In particular embodiments, the antigen is CD8 and the receptor is CD3. In further embodiments, the targeting component is an antibody specific for CD3 or the TCR or an associated molecule.

In specific embodiments, the B cell specific antigen is a viral antigen, a bacterial antigen, a tumor antigen, a self-antigen, and the like and the targeting component is an anti-BCR.

Each of the cell selector component, i.e., the peptide-MHC multimer and the B cell specific antigen, and the targeting component is associated with an oligonucleotide. In other words, each of the cell selector component and the targeting component can be bound to an oligonucleotide. As used herein, the phrase “bound to” refers to the binding (e.g., covalent binding, hybridization, etc.) of one structure to another structure, either directly or indirectly via a secondary structure. Thus, the cell selector component is bound to a first oligonucleotide, either by a direct bond or indirectly via a secondary structure, and the targeting component is bound to a second oligonucleotide, either by a direct bond or indirectly via a secondary structure.

A structure comprising a cell selector component or targeting component, and an oligonucleotide, which is bound to (e.g., covalently bound) to the cell selector component or targeting component, is referred to herein as a “PLA probe.” For example, in embodiments, a first PLA probe comprises a cell selector component bound (e.g., covalently bound) to a first oligonucleotide. In other embodiments, the first PLA probe comprises a first oligonucleotide bound to a secondary antibody that is specific for the cell selector component. Thus, in this embodiment, the first PLA probe comprises, the first oligonucleotide, the secondary antibody, and the cell selector component. In such embodiments, the first oligonucleotide is bound to the cell selector component when the secondary antibody contacts and binds to the cell selector component. In particular embodiments, a secondary antibody is specific for a tag on the cell selector component. Any suitable tag, such as a myc-tag, a his-tag, a fluorescent tag, such as for example, PE (phycoerythrin), FITC (fluorescein isothiocyanate), or APC (allophycocyanin) can be used.

Similarly, the second PLA probe comprises a second oligonucleotide. In embodiments, the second PLA probe comprises the second oligonucleotide bound (e.g., covalently bound) directly to the targeting component, such as a primary antibody, or antigen binding fragment thereof, a primary fusion protein, or a primary aptamer. In other embodiments, the second PLA probe comprises the second oligonucleotide bound (e.g., covalently bound) to a secondary antibody that is specific for the targeting component. In such embodiments, the second oligonucleotide is bound to the targeting component (e.g., a primary antibody) when the secondary antibody contacts and binds to the targeting component.

In particular embodiments, the first PLA probe comprises a cell selector component bound to a first oligonucleotide, and the second PLA probe comprises a targeting component (e.g., an antibody or an antigen binding fragment thereof) bound to a second oligonucleotide. In other specific embodiments, the first PLA probe comprises a first secondary antibody bound to the first oligonucleotide, and the second PLA probe comprises a second secondary antibody bound to the second oligonucleotide.

In specific embodiments, the first PLA probe comprises a cell selector component bound to a first oligonucleotide, and the second PLA probe comprises an anti-TCR antibody bound to a second oligonucleotide.

After the cells have been incubated with the probes, a ligation reagent is then introduced into the sample. A “ligation reagent” comprises a ligase that catalyzes a joining reaction between DNA molecules by forming a chemical bond). Any suitable ligation reagent can be used. For example, a ligation reagent comprising a protein ligase, a template independent ligase, a nucleic acid ligase, a chemical ligase, or a combination thereof. In embodiments, the ligation reagent comprises a protein ligase (e.g., T4 DNA ligase). In some embodiments, the ligation reagent comprises a template independent ligase (e.g., T4 RNA ligase 1 or 2). In further embodiments, the ligation reagent comprises a nucleic acid ligase (e.g., a ribozyme or a deoxyribozyme).

If the antigen on a cell (e.g., an antigen-specific T cell or B cell) stained with the cell selector component is in close proximity to the receptor (to which the targeting component binds), the ligation reagent ligates the first oligonucleotide and the second oligonucleotide to form a nucleic acid template.

In various embodiments, one or more labels (i.e., unique molecular identifiers) are bound to the amplification product, first PLA probe, the second PLA probe, or both. Any suitable label that would provide a distinguishing feature for the purposes of downstream quantification, sequencing, enrichment, and the like can be used. In some embodiments, the label(s) include a barcode (e.g., a nucleotide barcode). In some embodiments, the label comprises a heavy metal label. In some embodiments, the label comprises a fluorophore label. In still further embodiments, label comprises a DNA barcode labels, such as those described in Bentzen, et al. Nature Biotechnology, 2016, 34(10), 1037-1048.

Accordingly, the present disclosure comprises synthetic constructs comprising a cell selector component; a first oligonucleotide bound to the cell selector component; a second oligonucleotide bound to the first oligonucleotide; and a targeting component bound to the second oligonucleotide. As used herein, a “synthetic construct” refers to an artificially constructed structure that comprises a combination of peptides and oligonucleotides bound together.

In embodiments, the first oligonucleotide is bound to the cell selector component. In other embodiments, the first oligonucleotide is bound to a first secondary antibody that is bound to the cell selector component. In particular embodiments, the first secondary antibody is bound to a myc-tag, a his-tag, or a fluorescent tag on the cell selector component. In still other embodiments, the first oligonucleotide is bound to streptavidin. In some embodiments, the first oligonucleotide can be bound to any one of the four binding sites on streptavidin. In some embodiments, the second oligonucleotide is bound to the targeting component. In further embodiments, the second oligonucleotide is bound to a second secondary antibody that is bound to the targeting component. In particular embodiments, the cell selector component comprises a peptide-MHC multimer that can be a peptide-MHC tetramer. In particular embodiments, the cell selector component comprises a B cell specific antigen comprising viral- or bacterial-derived antigens, tumor antigens or self-antigens that could be proteins, carbohydrates, glycoproteins, lipids, nucleic acids, or other structures recognized by the BCR. In some embodiments, the targeting component comprises an antibody or antigen binding fragment thereof, a fusion protein, an aptamer, or a combination thereof, binding to a receptor. In embodiments, the receptor is CD3 or the TCR. In particular embodiments, the target cell is a T cell. In other embodiments, the receptor is CD79 or the BCR, or an associated molecule, and the target cell is a B cell. In specific embodiments, the construct further comprises a connector oligonucleotide bound to the first and second oligonucleotides.

In embodiments, the antigen is in close proximity if it is no more than 50 nm from the receptor. In some embodiments, the antigen is no more than 40 nm from the receptor. In further embodiments, the antigen is no more than 35 nm from the receptor. In further embodiments, the antigen is no more than 30 nm from the receptor. In specific embodiments, the antigen is no more than 25 nm from the receptor. Because of the close proximity of the receptor to the antigen, the cell selector component would also be in close proximity to the targeting component. In embodiments, the cell selector component is no more than 50 nm from the targeting component. In some embodiments, the cell selector component is no more than 40 nm from the targeting component. In further embodiments, the cell selector component is no more than 35 nm from the targeting component. In further embodiments, the cell selector component is no more than 30 nm from the targeting component. In specific embodiments, the cell selector component is no more than 25 nm from the targeting component.

In embodiments, the first oligonucleotide is bound to a particular area on the cell selector component, the second oligonucleotide is bound to a particular area on the second targeting component, or both. In such embodiments, the proximity of the first and second oligonucleotides can only be close enough when the cell selector component, the second targeting component, or both bind to the cell in a particular orientation.

One or more connector oligonucleotides are optionally added before, with, or after the ligation reagent. At least a portion of a connector oligonucleotide is complementary with at least a portion of the first oligonucleotide, the second oligonucleotide, or both. In such embodiments, the first oligonucleotide, the second oligonucleotide, and the connector oligonucleotide are ligated. The nucleic acid template is then amplified. Any suitable amplification method can be used, such as rolling circle amplification.

Accordingly, the present disclosure comprises methods for detecting a target cell in a sample, comprising: (a) contacting the target cell with a cell selector component for which the target cell is specific, the cell selector component being bound to a first oligonucleotide; (b) contacting the target cell with a targeting component specific for a receptor on the target cell, the targeting component being bound to a second oligonucleotide; (c) forming a nucleic acid template by ligating the first oligonucleotide and the second oligonucleotide; (d) binding a label (detection probe) incorporating a metal, fluorescent, and/or oligonucleotide barcode to the nucleic acid template, and (e) detecting the label on the target cell by mass cytometry, flow cytometry, sequencing and/or PCR. In various embodiments, methods of the present disclosure further comprise amplifying the nucleic acid template. Although described in the order shown above, the steps of the described methods can be performed in any suitable order. For example, the present disclosure further includes methods for detecting a target cell in a sample, comprising: (b) contacting the target cell with a targeting component specific for a receptor on the target cell, the targeting component being bound to a second oligonucleotide; (a) contacting the target cell with a cell selector component for which the target cell is specific, the cell selector component being bound to a first oligonucleotide; (c) forming a nucleic acid template by ligating the first oligonucleotide and the second oligonucleotide; (d) binding a label incorporating a metal, fluorescent, and/or oligonucleotide barcode to the nucleic acid template, and (e) detecting the label on the target cell by mass cytometry, flow cytometry, sequencing and/or PCR.

Any suitable tissue sample or call sample can be used in a method of the disclosure. For example, the tissue sample or cell sample can be isolated from a bodily fluid (e.g., blood products, bone marrow, peripheral blood mononuclear cells, etc.). In another example, the tissue sample is a tissue fragment, for example a biopsy or a tissue slide. In some embodiments, the cell sample is constituted by cells dissociated from tissue. In other embodiments, staining is performed on tissue slides in situ and detected using an imaging method. Methods for staining tissues using peptide-MHC multimers are described, for example, in Zhu, et al., Journal of Experimental Medicine, 2007, 204(3), 595-603. In various embodiments, the staining with the peptide-MHC multimers or the antigens occurs while the cells of the sample are alive. In alternate embodiments, the staining with the peptide-MHC multimers or the antigens occurs while the cells of the sample are fixed.

In embodiments, the amplification of the nucleic acid template comprises producing an amplification product. In some such embodiments, the method further comprises detecting the amplification product. Any suitable method for detecting the amplification product can be used. For example, flow cytometry, mass cytometry, time-of-flight mass spectrometry, single cell sequencing, bulk sequencing, or in situ imaging. Such methods are described in, e.g., Newell, et al, Nature Biotechnology, 2013, 31(7), 623-629; Bentzen, et al. Nature Biotechnology, 2016, 34(10), 1037-1048; and Davis, et al, Nature Reviews Immunology, 2011, 11(8), 551-558; which are incorporated by reference for their teachings regarding the same.

In various embodiments, detecting the amplification product comprises forming a labeled amplification product by contacting the amplification product with a probe comprising a label bound to an oligonucleotide that has a sequence that is substantially complementary to at least a portion of the amplification product. Any suitable label can be used, for example, a fluorescent label, a heavy metal tag, an oligonucleotide tag, and the like.

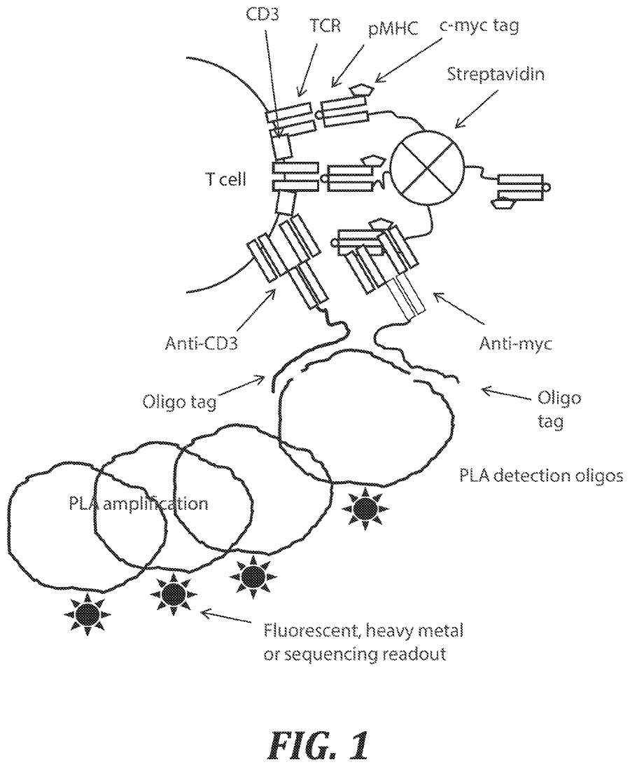

An illustration of an embodiment of a method and a synthetic construct of the disclosure is shown in FIG. 1. In this embodiment, a target T cell is contacted with a peptide-MHC multimer loaded with a peptide antigen for which the TCR is specific. Each monomer of the peptide-MHC multimer comprises a myc-tag, a his-tag, or a fluorescent tag, and the monomers are multimerized with streptavidin. A secondary anti-myc, anti-his, or anti-fluorochrome antibody bound to an oligonucleotide contacts the targeting component and binds to the myc-tag, to the his-tag, or to the fluorescent tag. The T cell is also contacted with an anti-CD3 antibody that is bound to a second oligonucleotide. PLA detection oligonucleotides are then amplified to create PLA products, which are labeled, e.g., a fluorescent label, a heavy metal label, an oligonucleotide label, etc.

It is contemplated that the peptide-MHC multimer of any of the embodiments described herein be replaced with an antigen. Any suitable antigen could be used. For example, a viral surface protein, protein complex, or other macromolecule. In some embodiments, the antigen is a virus or a microbe.

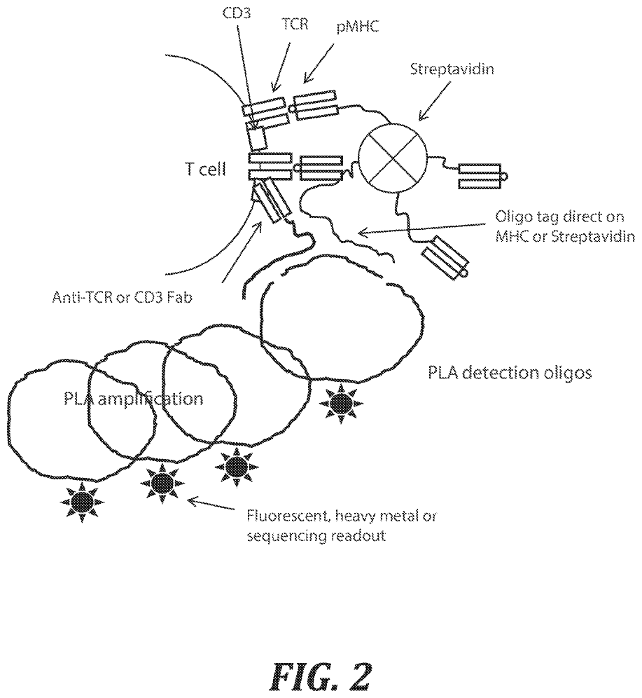

An illustration of an alternate embodiment of a method and a synthetic construct of the disclosure is shown in FIG. 2. In this embodiment, a target T cell is contacted with a peptide-MHC multimer loaded with a peptide antigen for which the TCR is specific. The monomers are multimerized with streptavidin, and an oligonucleotide is bound to a monomer or the streptavidin. The T cell is also contacted with an anti-TCR or anti-CD3 Fab that is bound to a second oligonucleotide. PLA detection oligonucleotides are then amplified to create PLA products, which are labeled, e.g., a fluorescent label, a heavy metal label, an oligonucleotide tag, etc.

An illustration of a further embodiment of a method and a synthetic construct of the disclosure is shown in FIG. 3. In this embodiment, a target B cell is contacted with an antigen of the BCR. The antigen is bound to an oligonucleotide. The B cell is also contacted with an anti-BCR or anti-CD79 Fab that is bound to a second oligonucleotide. PLA detection oligonucleotides are then amplified to create PLA products, which are labeled, e.g., a fluorescent label, a heavy metal label, an oligonucleotide tag, etc.

In embodiments, the amplification product is used in a sequencing method. In embodiments, this is performed by using a detection probe that includes appropriate sequencing or PCR adapters and molecular barcode sequences. In further embodiments, the amplification product is used to separate the target cells. For example, a label, such as a fluorophore, could be used to sort the target cells using flow cytometry and fluorescence-activated cell sorting (FACS). In other embodiments, the amplification product is used to isolate the target cells. As used herein, “isolate” or the like refers to the separation of a cell, or a population of cells, from its original environment, i.e., the environment of the isolated cells is substantially free of at least one component as found in the environment in which the “un-isolated” reference cells exist. “Isolated” includes a cell that is removed from some or all components as it is found in its natural environment, for example, isolated from a tissue or biopsy sample. “Isolated” also includes a cell that is removed from at least one, some or all components as the cell is found in non-naturally occurring environments, for example, isolated from a cell culture or cell suspension. Therefore, an isolated cell is partly or completely separated from at least one component, including other substances, cells, or cell populations, as it is found in nature or as it is grown, stored, or subsisted in non-naturally occurring environments. Isolated cells can be obtained from separating the desired cells, or populations thereof, from other substances or cells in the environment, or from removing one or more other cell populations or subpopulations from the environment. Specific examples of isolated cells include partially pure cell compositions, substantially pure cell compositions and cells cultured in a medium that is non-naturally occurring.

In still further embodiments, the amplification product is used to purify the cells. As used herein, “purify” or the like refers to the process of increasing the purity of a sample. For example, the purity can be increased to at least 50%, 60%, 70%, 80%, 90%, 95%, 99%, or 100%.

In some embodiments, the isolated or purified cells can then be further processed to sequence the TCR or BCR, the receptors that are responsible for binding to the antigen.

In some embodiments, the TCR or BCR receptors can be sequenced using single cell sequencing methods. Single cell sequencing can be performed according to any method well-known to one of ordinary skill in the art. For example, the method for single cell sequencing can include, but is not limited to, sorting, mixing, and loading the cells to be sequenced on to 10× genomics K chips for single cell encapsulation. In some embodiments, the library preparations for feature barcode and VDJ can be performed subsequentially and sequenced using next-generation sequencing (NGS). In some embodiments, the sequencing results of VDJ library can be analysed with Cellranger for TCR alignment, VDJ annotation and read counting for each cell. In still other embodiments, the feature barcode library can be analysed in parallel for counting a certain sequence in each cell. In still other embodiments, the unique clonotypes can be searched and matched to known sequences in a database.

In some embodiments, the TCR or BCR receptors can be sequenced using single cell PCR. Single cell PCR can be performed according to any method well-known to one of ordinary skill in the art. For example, the method for single cell PCR can include, but is not limited to, targeted PCR of TCR alpha chain and beta chain for each sorted cell and the resulting PCR product can be sequenced with sanger sequencing. The sequencing results can be blasted using IgBlast for alignment to CDR3 regions and annotation of VDJ names.

In embodiments, the amplification product is used to enrich the cells in a composition. In certain embodiments, a composition that is enriched for target cells as disclosed herein is also depleted for cells that are not target cells. “Enriched” and “depleted” as used herein to describe amounts of cell types in a mixture refer, respectively, to the subjecting of a mixture or sample of the cells to a process or step (e.g., sorting) which results in an increase in the number or relative amount of the “enriched” type, in certain embodiments, and a decrease in the number or relative amount of the “depleted” cells relative to the mixture or sample prior to the process or step.

In various embodiments, the synthetic construct remains bound to the cell throughout the processing. In other embodiments, at least a portion of the synthetic construct is removed. For example, after isolation or purification of the cells. In some such embodiments, at least a portion of the synthetic construct is removed via cleavable linkers bound to the oligonucleotides.

In some embodiments, more than one pair of cell selector component and targeting component can be used. In some such embodiments, more than one pair of corresponding PLA probes can be used. In such embodiments, a different label can be used for each pair of corresponding PLA probes. In some embodiments, a different fluorophore label can be used for each pair of corresponding PLA probes. In other embodiments, the different labels for each pair of corresponding PLA probes comprise at least one heavy metal label. In some embodiments, the different labels for each pair of corresponding PLA probes comprise a combination of fluorophore labels and heavy metal labels. In still further embodiments, the different labels are DNA barcode labels, such as those described in Bentzen, et al. Nature Biotechnology, 2016, 34(10), 1037-1048.

The present disclosure further provides kits that can be used to perform the methods described herein. Such kits for processing (e.g., detecting, separating, isolating, enriching, or purifying) target cell(s) in a sample comprise: (a) a cell selector component for which the target cell is specific, the cell selector component being bound to a first oligonucleotide; (b) a targeting component specific for a second antigen on the target cell, the targeting component being bound to a second oligonucleotide; (c) a ligation reagent; and (d) written instructions for using the cell selector component and the targeting component to detect the target cell.

Any suitable ligation reagent can be included. For example, a protein ligase, a template independent ligase, a nucleic acid ligase, a chemical ligase, or a combination thereof. In embodiments, the ligation reagent comprises a protein ligase. In other embodiments, the ligating reagent comprises a chemical ligase. In further embodiments, the ligating reagent comprises a nucleic acid ligase. In still further embodiments, the ligating reagent comprises a template independent ligase.

In some embodiments, a kit further comprises a connector oligonucleotide. In further embodiments, a kit further comprises a probe comprising a fourth oligonucleotide and a label. In some such embodiments, the label is a fluorescent label. In other embodiments, the label is a heavy metal tag.

In various embodiments, the written instructions can be in the form of printed instructions provided within the kit, or the written instructions can be printed on a portion of the container housing the kit. Written instructions can be in the form of a sheet, pamphlet, brochure, CD-ROM, or computer-readable device, or can provide directions to locate instructions at a remote location, such as a website. The written instructions can be in English and/or in a national or regional language.

Kits can further comprise one or more syringes, ampules, vials, tubes, tubing, facemask, a needleless fluid transfer device, an injection cap, sponges, sterile adhesive strips, Chloraprep, gloves, and the like. Variations in contents of any of the kits described herein can be made. In various embodiments, the content of the kit is provided in a compact container.

### EMBODIMENTS

Embodiment 1. A method for detecting a target cell in a sample, comprising: contacting the target cell with a cell selector component for which the target cell is specific, the cell selector component being bound to a first oligonucleotide; contacting the target cell with a targeting component specific for a receptor on the target cell, the targeting component being bound to a second oligonucleotide; and forming a nucleic acid template by ligating the first oligonucleotide and the second oligonucleotide.

Embodiment 2. The method of embodiment 1, wherein the first oligonucleotide is directly bound to the cell selector component.

Embodiment 3. The method of embodiment 1 or 2, wherein the second oligonucleotide is directly bound to the targeting component.

Embodiment 4. The method of any one of embodiments 1-3, wherein the first oligonucleotide is bound to a first secondary antibody that is bound to the cell selector component.

Embodiment 5. The method of any one of embodiments 1-4, wherein the first secondary antibody is bound to a myc-tag, a his-tag, or a fluorescent tag on the cell selector component.

Embodiment 6. The method of any one of embodiments 1-5, wherein the second oligonucleotide is bound to a second secondary antibody that is bound to the targeting component.

Embodiment 7. The method of any one of embodiments 1-6, wherein the cell selector component comprises a peptide-MHC multimer.

Embodiment 8. The method of any one of embodiments 1-7, wherein the peptide-MHC multimer comprises a peptide-MHC tetramer Embodiment 9. The method of any one of embodiments 1-8, wherein the peptide-MHC multimer comprises streptavidin or dextran.

Embodiment 10. The method of any one of embodiments 1-9, wherein the first oligonucleotide is bound to streptavidin or dextran.

Embodiment 11. The method of any one of embodiments 1-10, wherein the cell selector component comprises a B cell specific antigen.

Embodiment 12. The method of any one of embodiments 1-11, wherein the targeting component comprises an antibody or antigen binding fragment thereof, a fusion protein, an aptamer, or a combination thereof.

Embodiment 13. The method of any one of embodiments 1-12, wherein the receptor is CD3 or the T cell receptor (TCR).

Embodiment 14. The method of any one of embodiments 1-13, wherein the target cell is a T cell.

Embodiment 15. The method of any one of embodiments 1-14, wherein the receptor is CD79 of the B cell receptor (BCR).

Embodiment 16. The method of any one of embodiments 1-15, wherein the target cell is a B cell.

Embodiment 17. The method of any one of embodiments 1-16, wherein the cell selector component is no more than 50 nanometers (nm) from the targeting component.

Embodiment 18. The method of any one of embodiments 1-17, wherein the cell selector component is no more than 40 nm from the targeting component.

Embodiment 19. The method of any one of embodiments 1-18, wherein the cell selector component is no more than 35 nm from the targeting component.

Embodiment 20. The method of any one of embodiments 1-19, wherein the cell selector component is no more than 30 nm from the targeting component.

Embodiment 21. The method of any one of embodiments 1-20, wherein the cell selector component is no more than 25 nm from the targeting component.

Embodiment 22. The method of any one of embodiments 1-21, further comprising contacting the first and second oligonucleotides with a connector oligonucleotide.

Embodiment 23. The method of any one of embodiments 1-22, further comprising amplifying the nucleic acid template.

Embodiment 24. The method of any one of embodiments 1-23, wherein the amplifying the nucleic acid template comprises rolling circle amplification.

Embodiment 25. The method of any one of embodiments 1-24, wherein the amplifying the nucleic acid template comprises producing an amplification product.

Embodiment 26. The method of any one of embodiments 1-25, further comprising detecting the amplification product.

Embodiment 27. The method of any one of embodiments 1-26, wherein the detecting the amplification product comprises forming a labeled amplification product by contacting the amplification product with a probe comprising a fourth oligonucleotide and a label, the fourth oligonucleotide having a sequence that is substantially complementary to at least a portion of the amplification product.

Embodiment 28. The method of any one of embodiments 1-27, wherein the label is a fluorescent label.

Embodiment 29. The method of any one of embodiments 1-28, wherein the label is a heavy metal tag.

Embodiment 30. The method of any one of embodiments 1-29, wherein the label is an oligonucleotide tag or a DNA barcode.

Embodiment 31. The method of any one of embodiments 1-30, wherein the detecting the amplification product comprises imaging the labeled amplification product using flow cytometry.

Embodiment 32. The method of any one of embodiments 1-31, wherein the detecting the amplification product comprises using time-of-flight mass spectrometry.

Embodiment 33. The method of any one of embodiments 1-32, wherein the detecting the amplification product comprises using sequencing or PCR.

Embodiment 34. The method of any one of embodiments 1-33, wherein the ligating the first oligonucleotide and the second oligonucleotide comprises using a protein ligase.

Embodiment 35. The method of any one of embodiments 1-34, wherein the ligating the first oligonucleotide and the second oligonucleotide comprises using a chemical ligase.

Embodiment 36. The method of any one of embodiments 1-35, wherein the ligating the first oligonucleotide and the second oligonucleotide comprises using a nucleic acid ligase.

Embodiment 37. The method of any one of embodiments 1-36, wherein the sample is a tissue sample or a cell sample.

Embodiment 38. The method of any one of embodiments 1-37, further comprising sequencing the target cell receptor.

Embodiment 39. A kit for detecting a target cell in a sample comprising: a cell selector component for which the target cell is specific, the cell selector component being bound to a first oligonucleotide; a targeting component specific for a second antigen on the target cell, the targeting component being bound to a second oligonucleotide; a ligation reagent; and written instructions for using the cell selector component and the targeting component to detect the target cell.

Embodiment 40. The kit of embodiment 39, further comprising a connector oligonucleotide.

Embodiment 41. The kit of embodiment 39 or 40, further comprising a probe comprising a fourth oligonucleotide and a label.

Embodiment 42. The kit of any one of embodiments 39-41, wherein the label is a fluorescent label.

Embodiment 43. The kit of any one of embodiments 39-42, wherein the label is a heavy metal tag.

Embodiment 44. The kit of any one of embodiments 39-43, wherein the label is an oligonucleotide tag or a DNA barcode.

Embodiment 45. The kit of any one of embodiments 39-44, wherein the ligation reagent comprises a protein ligase.

Embodiment 46. The kit of any one of embodiments 39-45, wherein the ligation reagent comprises a chemical ligase.

Embodiment 47. The kit of any one of embodiments 39-46, wherein the ligation reagent comprises a nucleic acid ligase.

Embodiment 48. A synthetic construct, comprising: a cell selector component; a first oligonucleotide bound to the cell selector component; a second oligonucleotide bound to the first oligonucleotide; and a targeting component bound to the second oligonucleotide.

Embodiment 49. The synthetic construct of embodiment 48, wherein the first oligonucleotide is bound directly to the cell selector component.

Embodiment 50. The synthetic construct of embodiment 48 or 49, wherein the second oligonucleotide is directly bound to the targeting component.

Embodiment 51. The synthetic construct of any one of embodiments 48-50, wherein the first oligonucleotide is bound to a first secondary antibody that is bound to the cell selector component.

Embodiment 52. The synthetic construct of any one of embodiments 48-51, wherein the first secondary antibody is bound to a myc-tag, a his-tag, or a fluorescent tag on the cell selector component.

Embodiment 53. The synthetic construct of any one of embodiments 48-52, wherein the second oligonucleotide is bound to a second secondary antibody that is bound to the targeting component.

Embodiment 54. The synthetic construct of any one of embodiments 48-53, wherein the cell selector component comprises a peptide-MHC multimer.

Embodiment 55. The synthetic construct of any one of embodiments 48-54, wherein the peptide-MHC multimer comprises a peptide-MHC tetramer

Embodiment 56. The synthetic construct of any one of embodiments 48-55, wherein the peptide-MHC multimer comprises streptavidin or dextran.

Embodiment 57. The synthetic construct of any one of embodiments 48-56, wherein the first oligonucleotide is bound to streptavidin or dextran.

Embodiment 58. The synthetic construct of any one of embodiments 48-57, wherein the cell selector component comprises a B cell specific antigen.

Embodiment 59. The synthetic construct of any one of embodiments 48-58, wherein the targeting component comprises an antibody or antigen binding fragment thereof, a fusion protein, an aptamer, or a combination thereof.

Embodiment 60. The synthetic construct of any one of embodiments 48-59, wherein the receptor is CD3 or the T cell receptor (TCR).

Embodiment 61. The synthetic construct of any one of embodiments 48-60, wherein the target cell is a T cell.

Embodiment 62. The synthetic construct of any one of embodiments 48-61, wherein the receptor is CD79 or the B cell receptor (BCR).

Embodiment 63. The synthetic construct of any one of embodiments 48-62, wherein the target cell is a B cell.

Embodiment 64. The synthetic construct of any one of embodiments 48-63, further comprising a connector oligonucleotide bound to the first and second oligonucleotides.

### EXAMPLES

The following examples are set forth so as to provide those of ordinary skill in the art with a complete disclosure and description of how to make and use the present invention and are not intended to limit the scope of what the inventors regard as their invention nor are they intended to represent that the experiments below are all or the only experiments performed.

### Example 1

### General Reagents and Materials

Human blood samples were collected from donors. Peripheral blood mononuclear cells (PBMCs) were obtained by Ficoll density centrifugation. 16% Paraformaldehyde (PFA, 28906) and polyclonal rabbit anti-PE antibody (PA5-35006) were from ThermoFisher Scientific. Polyclonal rabbit anti-his tag (A00174) was from Genscript. PBMCs transduced with MAGE-A1 and MAGE-A4-specific TCRs were produced in-house. SARS-CoV-2 spike antigen complex were prepared from SARS-CoV-2 RBD B Cell Analysis Kit (Miltenyi, 130-128-032). Duolink® in situ PLA probe maker kit (DUO92009, DUO92010), Duolink® flowPLA secondary abs kits (DUO92002, DUO92005), Duolink® flowPLA detection kits (DUO94002, DUO94004) were from MilliporeSigma. Unfold PLA kits and probe conjugation kits were from Navinci. Rabbit anti-hCD3 (ab135372) was from Abcam. Mouse anti-hCD3 (300438), mouse anti-hTCRa/b-BV510 (306733) and mouse anti-hTCRα/b (306702) were from Biolegend. Anti-human CD19-Pacific Blue (302223), Purified anti-human 79a (333502) and anti-human 79b (341404) were from Biolegend.

### Example 2

### PLA Assay

This Example describes a method to increase the specificity of antigen-specific tetramer staining based on proximity ligation assay (PLA). This method uses the fact that CD3 is close enough to the TCR-MHC tetramer complex to generate a PLA signal (FIG. 4A).

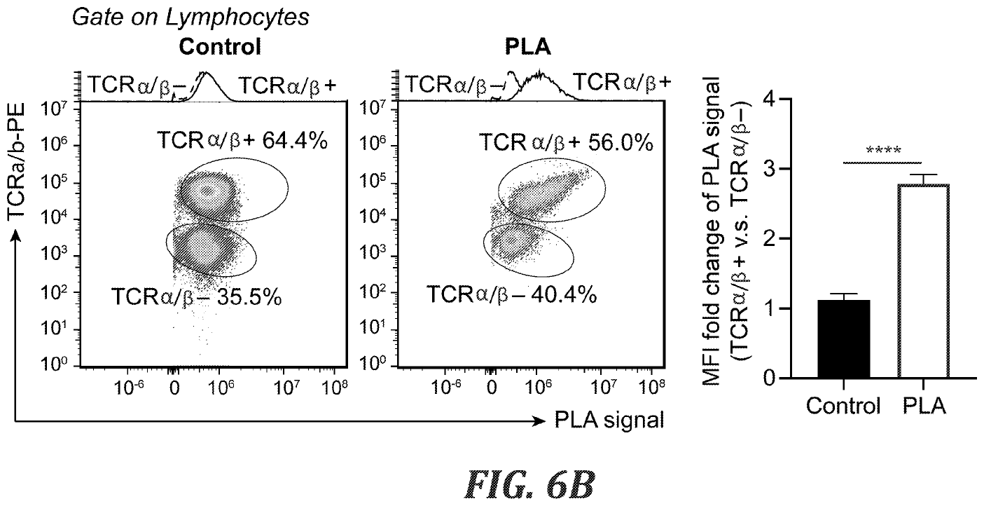

To begin, experiments were designed to demonstrate that CD3 and TCR are close enough to generate a PLA signal. Peripheral blood mononuclear cells (PBMCs) were stained with anti-CD3 and anti-TCRα/β-BV510 antibodies followed by staining with secondary antibodies bearing PLA oligo probes targeting anti-CD3 and anti-TCRα/β-BV510 respectively. Flow cytometric analysis showed TCRα/β+ population exhibited significantly higher PLA signal compared with TCRα/β− population (FIG. 4). In another experiment, PLA PLUS and MINUS probes were directly conjugated to anti-CD3 and anti-TCRα/β antibodies, respectively. After PLA, a distinct PLA+ population was observed which was 58.4% of total lymphocytes and mainly contained CD4+ or CD8+, showing the specificity of the PLA staining (FIG. 5). To further confirm the specificity of PLA, PBMCs were stained with an anti-TCRα/β-PE antibody, followed by the staining with an anti-CD3 antibody bearing a PLUS oligo probe (anti-CD3-PLUS) and anti-PE antibody bearing a MINUS probe (anti-PE-MINUS). After PLA amplification, flow cytometry showed that PE+ population exhibited 3-fold stronger PLA signal than that of PE− population. In contrast, cells without antibody-probes only showed background PLA signal (FIG. 6).

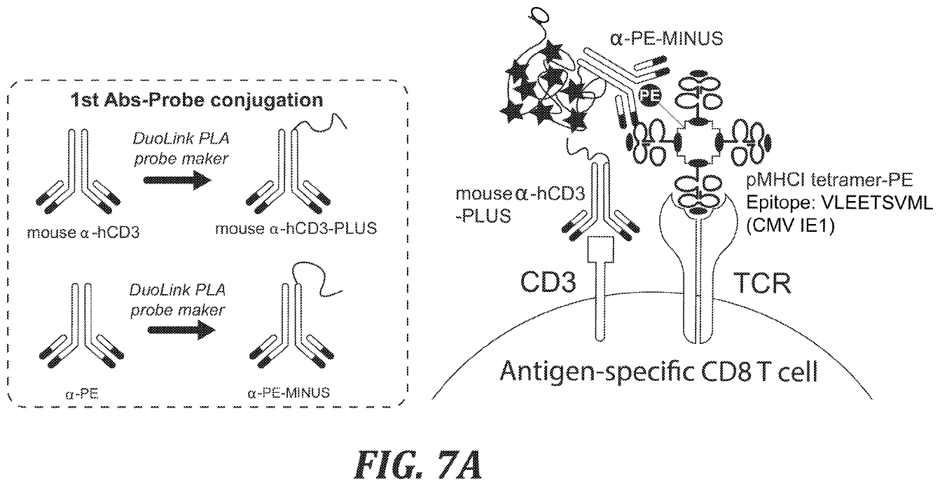

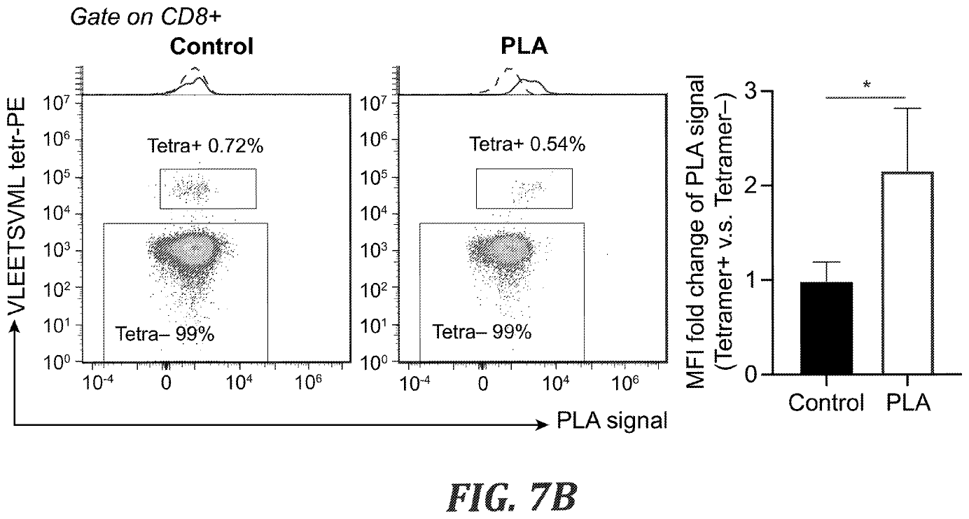

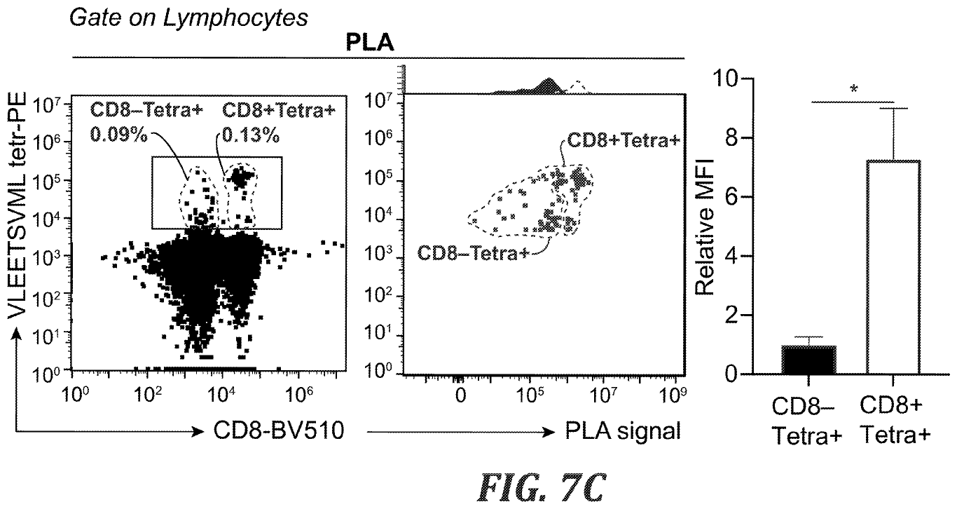

To validate that PLA also works on tetramer staining, cells were stained with a PE-labelled p:MHCI tetramer (epitope: CMV IE1 VLEETSVML) and then anti-CD3-PLUS and anti-PE-MINUS antibodies were introduced. After PLA amplification, we observed the specific PLA signal on CD8+Tetramer+ population (FIGS. 7A and 7B), showing significantly stronger PLA signal than that of CD8+Tetramer− population.

In another experiment, we observed that CD8-Tetramer+(0.09% in total lymphocytes, presumably background due to low level of non-specific tetramer staining) and CD8+Tetramer+(0.13% in total lymphocytes) co-exists in PBMCs (FIG. 7C). After PLA, CD8+Tetramer⁺ cells showed 8-fold stronger PLA signal than that of CD8-Tetramer+ population (i.e., non-antigen-specific tetramer staining), showing that PLA increases the specific tetramer signal.

### Methods

### Performing Proximity Ligation Assay (PLA) Targeting CD3 and TCR Using Human PBMCs Based on Secondary Antibody-Probes Using Duolink Flow PLA Kit.

PBMCs from donor SDBB025 (0.2 M cells per well) were seeded in a 96-well plate. After treating with blocking buffer (Duolink® flowPLA kit) cells were stained with rabbit anti-hCD3 (SP162, 1:100) and mouse anti-hTCRα/b-BV510 (IP26, 1:100) for 1 hour at 4° C. After washing twice with PBS, cells were fixed with 2% PFA for 30 min at room temperature. Then the cells were incubated with Duolink® anti-rIgG-PLUS and Duolink® anti-mIgG-MINUS for 1 hour at 37° C., followed by ligation (30 min, 37° C.), amplification (200 min, 37° C.) and detection (30 min, 37° C.) according to Duolink® flowPLA user manual, which can be found on the MilliporeSigma website.

### General Procedure for Conjugating PLA Probes to Antibodies Using Duolink In Situ PLA Probe Maker Kit.

The conjugation of PLA probes to antibodies was performed according to Duolink® in situ PLA probe maker manual. In brief, 2 μL conjugation reagent was added to 20 μL target antibody (1 mg/mL). After gentle pipetting, the mixture was added to a vial containing lyophilized PLA probe (PLUS or MINUS) and the reaction mixture was left at room temperature for 16 hours. Then 2 μL stop reagent was added to quench the reaction for 30 min at room temperature. Then 24 μL storage solution was added, and the resulting antibody-probe was stored at 4° C.

### Performing PLA Targeting CD3 and TCR Using Human PBMCs Based on Primary Antibody-Probes Using Duolink Flow PLA Kit.

Duolink® PLUS and MINUS oligo probes were introduced to mouse anti-hCD3 (UTCH1) and mouse anti-hTCRα/D (IP26) respectively using Duolink® probe maker kit. PBMCs (0.2 M cells per well) were seeded in a 96-well plate. Cells were fixed with 2% paraformaldehyde (PFA) for 30 min at room temperature. After treating with blocking buffer (Duolink® flowPLA kit) cells were stained with mouse anti-hCD3-PLUS (1:100) and mouse anti-hTCRα/β-MINUS (1:100) for 1 hour at room temperature. Then cells were subjected to ligation (30 min, 37° C.), amplification (200 min, 37° C.) and detection (30 min, 37° C.) according to the user manual. After being stained with anti-hCD4-SB600 (SK3, 1:100) and anti-hCD8-SB780 (1:100), cells were analyzed by flow cytometry.

Performing PLA Targeting CD3 and TCR Using Human PBMCs with Anti-hCD3-PLUS and Anti-PE-MINUS (Duolink Flow PLA Kit).

Duolink® PLUS and MINUS oligo probes were introduced to mouse anti-hCD3 (UTCH1) and anti-PE (polyclonal) respectively using Duolink® probe maker kit. PBMCs (0.2 M cells per well) were seeded in a 96-well plate. Cells were treated with anti-hTCRα/β-PE (IP26, 1:100) for 30 min, followed by fixation with 2% paraformaldehyde (PFA) for 30 min at room temperature. After treating with blocking buffer (Duolink® flowPLA kit), cells were treated with mouse anti-hCD3-PLUS (1:100) and mouse anti-PE-MINUS (1:100) for 1 hour at room temperature. Then cells were subjected to ligation (30 min, 37° C.), amplification (200 min, 37° C.) and detection (30 min, 37° C.) according to the user manual.

Performing PLA Targeting CD3 and p:MHCI Tetramer Using Human PBMCs with Anti-hCD3-PLUS and Anti-PE-MINUS (Duolink Flow PLA Kit).

Duolink® PLUS and MINUS oligo probes were introduced to mouse anti-hCD3 (UTCH1) and anti-PE (polyclonal) respectively using Duolink® probe maker kit. PBMCs (0.2 M cells per well) were seeded in a 96-well plate. Cells were treated with p:MHCI tetramer (VLEETSVML, 1:25) for 30 min at room temperature, followed by fixation with 2% paraformaldehyde (PFA) for 30 min at room temperature. After treating with blocking buffer (Duolink® flowPLA kit), cells were stained with mouse anti-hCD3-PLUS (1:100) and mouse anti-PE-MINUS (1:100) for 1 hour at room temperature. Then cells were subjected to ligation (30 min, 37° C.), amplification (200 min, 37° C.) and detection (30 min, 37° C.) according to the user manual.

### Example 3

### Unfold PLA Assay for Antigen Specific T Cells

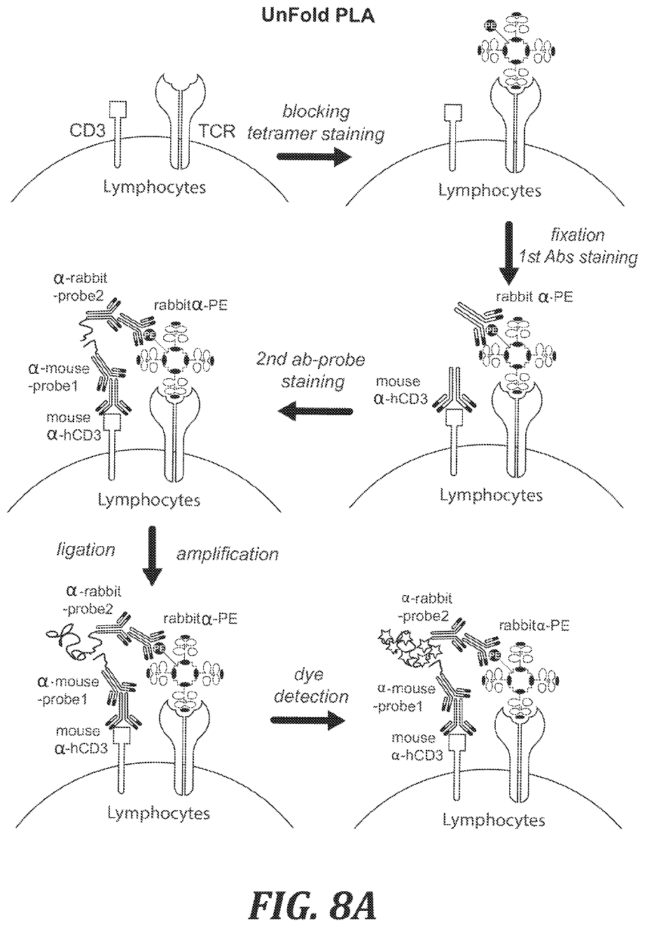

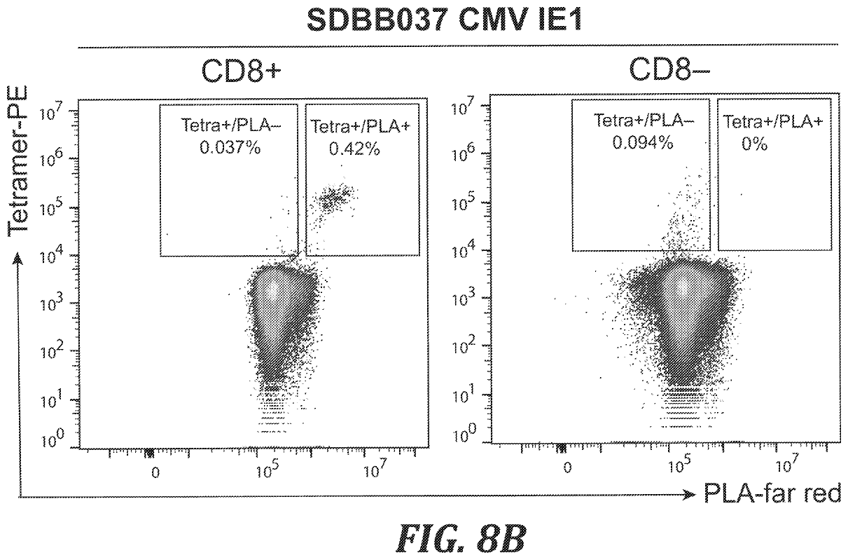

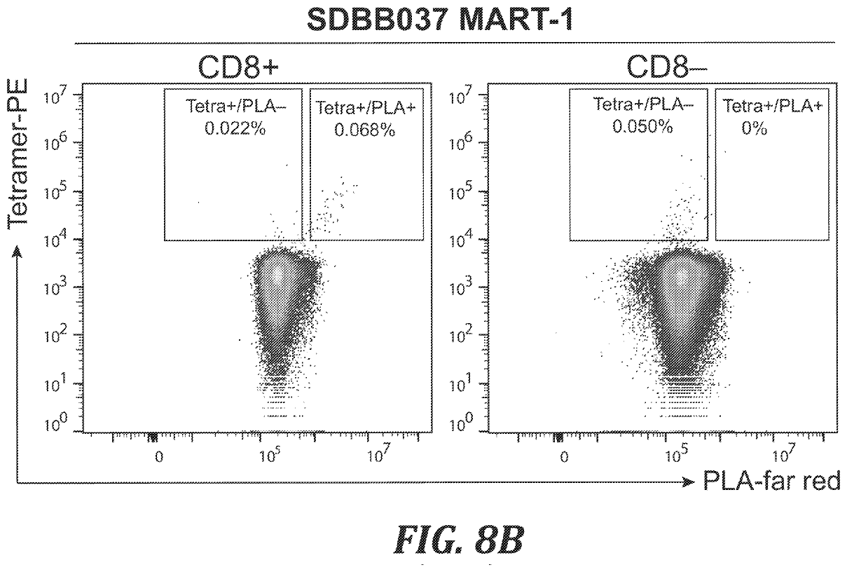

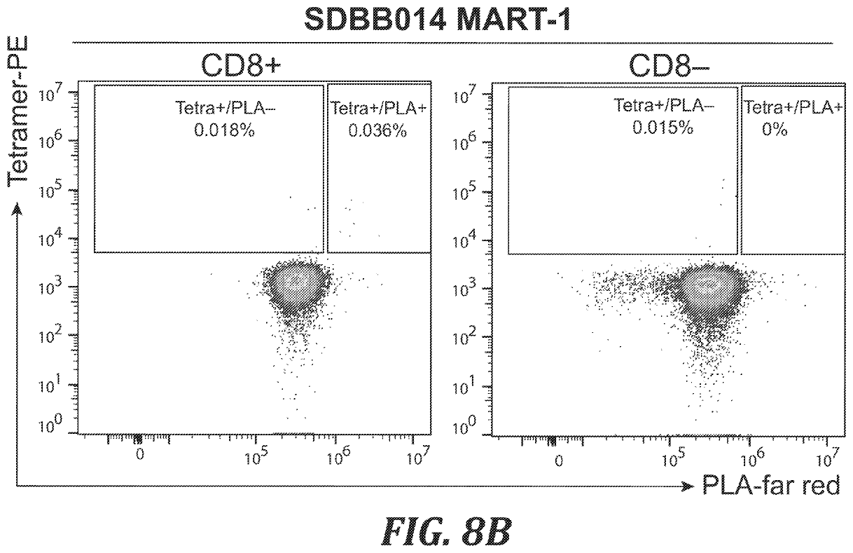

This Example describes a method to increase the specificity of antigen-specific tetramer staining based on the Unfold proximity ligation assay (PLA), which increases the maximum cell number per reaction and enables application in mass cytometry and sequencing. FIG. 8A illustrates the experimental protocol.

To begin, after being stained with tetramer bearing PE, cells were treated with mouse anti-human CD3 and rabbit anti-PE antibodies, followed by secondary antibody-probes targeting the corresponding primary antibodies. After ligation and amplification, a detection oligo bearing a far-red dye Atten647 were used to generate the PLA signal. As shown in FIG. 8B, PBMCs from SDBB037 were stained with CMV IE1 tetramer-PE. After Unfold PLA, ˜92% tetramer+ cells showed distinctive PLA signal with minimal background. Notably, Unfold PLA works well for the detection of tumor-specific T cells. 76% tetramer+ T cells from SDBB037 had a PLA signal when MART-1 tetramer was used, suggesting a substantial amount (˜24%) of tetramer+ T cells were false tumor-specific hits. Similarly, only 67% tetramer+ T cells from SDBB014 were showing PLA signal with MART-1 tetramer staining.

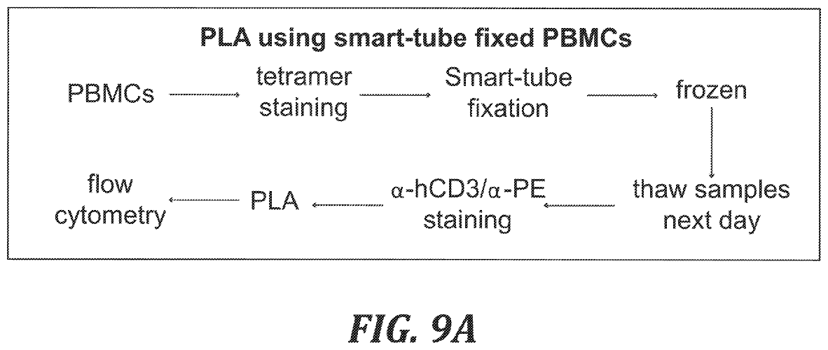

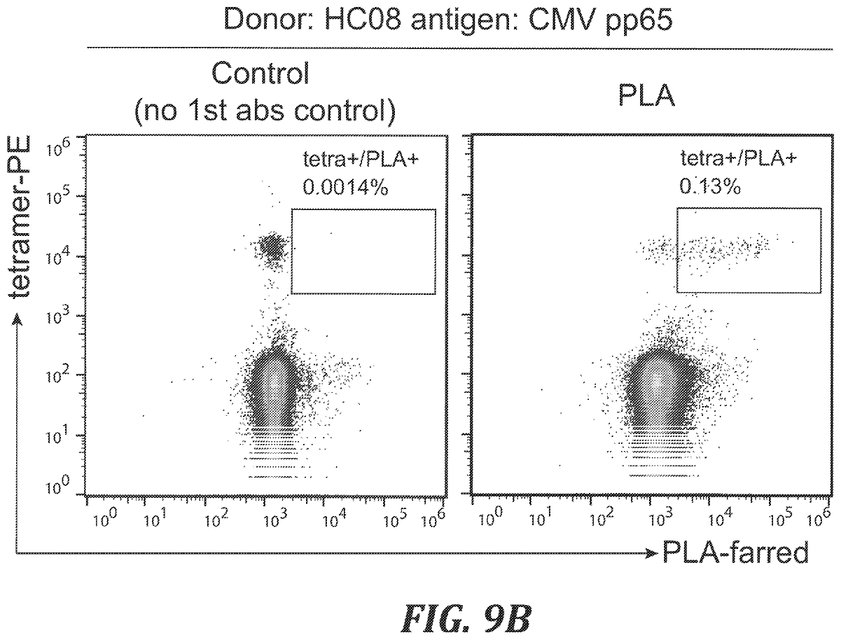

Notably, PLA is compatible with different fixation methods. As shown in FIG. 9, PBMCs from donor HC08 were stained with the tetramer bearing CMV pp65 peptide and then fixed/preserved using commercial Smart-tubes. After thawing, the Smart-tube fixed PBMCs could be directly subjected to PLA procedure to achieve a strong and specific PLA signal. This application allows our PLA protocol to be more flexible and be applied to different types of samples.

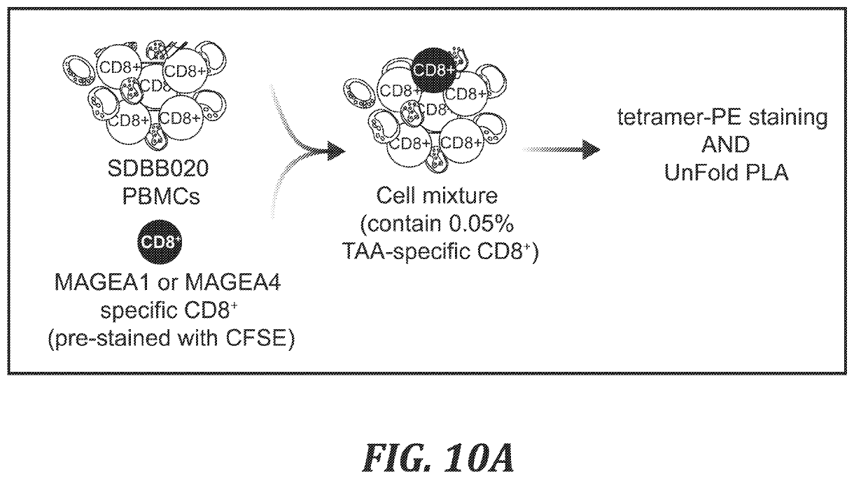

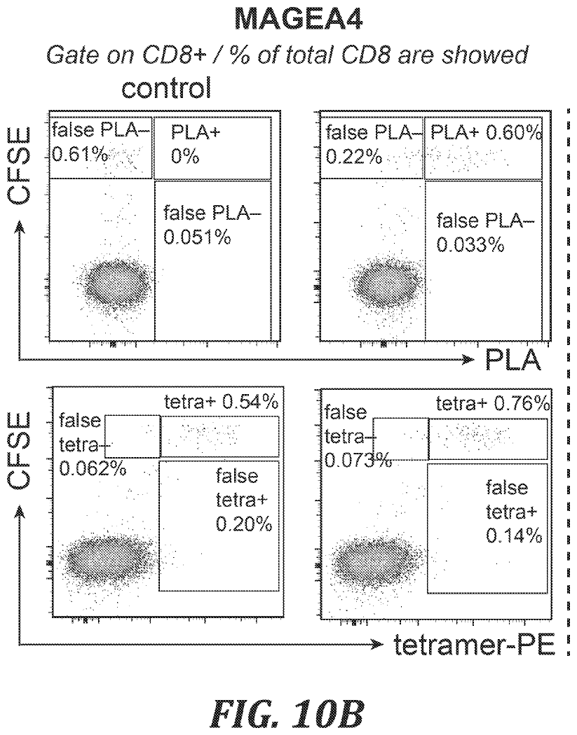

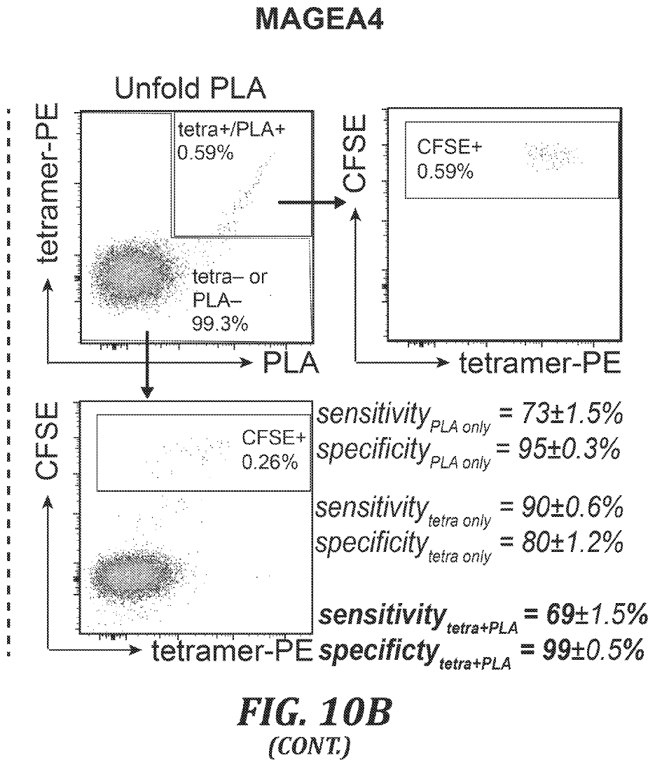

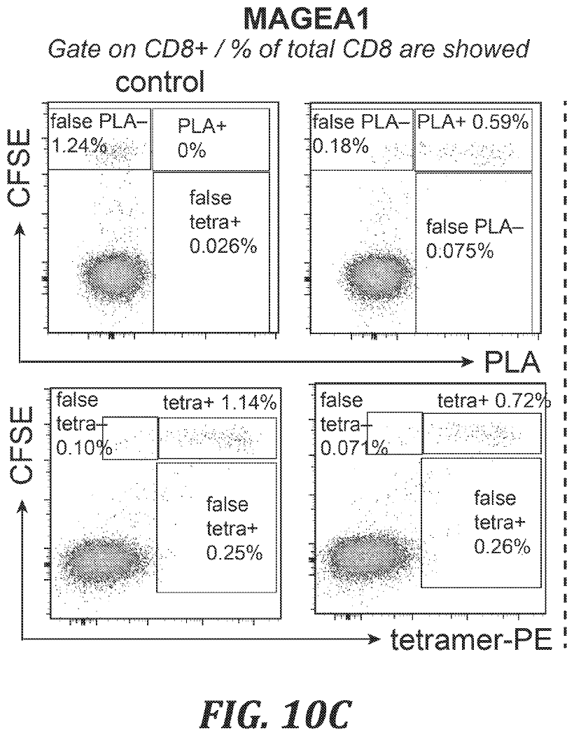

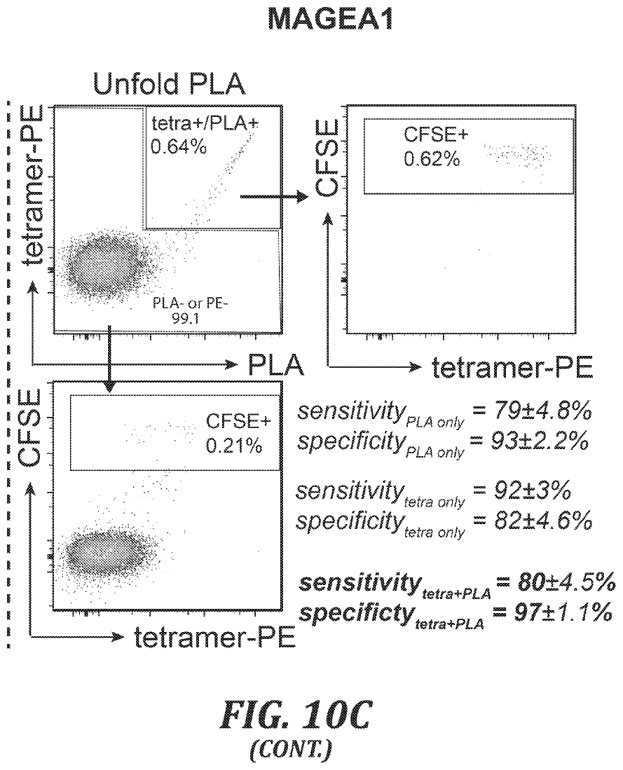

To further prove and evaluate if PLA can improve the specificity of antigen-specific T cells identification, the experiment shown in FIG. 10A was performed. CD8+ T cells transduced with MAGE-A1 or MAGE-A4-specific TCRs were pre-stained with carboxyfluorescein succinimidyl ester (CFSE) and mixed with PBMCs from healthy donor SDBB020 at the percentage of 0.05%. Then tetramer staining and Unfold PLA were performed. According to the signal of CFSE, tetramer-PE and PLA, the sensitivity and specificity of the antigen specific identification could be calculated and compared. As shown in FIG. 10B, the detection of MAGE-A4-specific CD8 T cells using tetramer showed a sensitivity of 90±0.6% and a specificity of 80±1.25%. In comparison, the detection solely relying on PLA reached a significantly higher specificity at 95±0.3% but a slightly lower sensitivity at 73±1.5%. Notably, the detection of MAGE-A4-specific T cells with a combination of tetramer staining and PLA could reach ˜100% specificity and ˜70% sensitivity, demonstrating how PLA can help improve the accuracy to identify antigen-specific T cells. The experiment using MAGE-A1-specific T cells showed similar results: tetramer staining with PLA reached −˜97% specificity and −˜80% sensitivity.

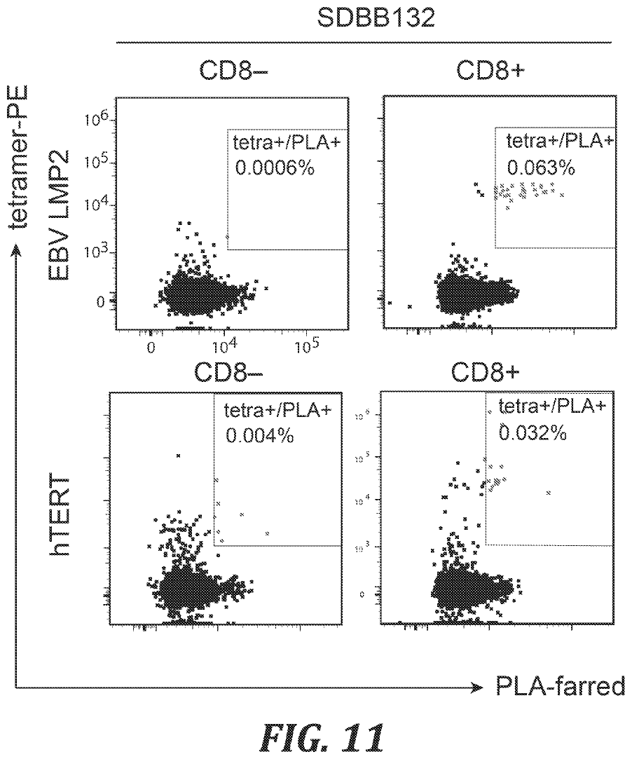

In another example shown in FIG. 11, SDBB132 has been identified to have EBV LMP2 and hTERT specific CD8 T cells. However, compared with the EBV LMP2 tetramer staining, hTERT tetramer showed significantly high background, which made it difficult to set a proper gate to identify real hTERT specific CD8 T cells. However, in combination with PLA, hTERT specific CD8 T cells could be distinguished according to both tetramer signal and PLA signal.

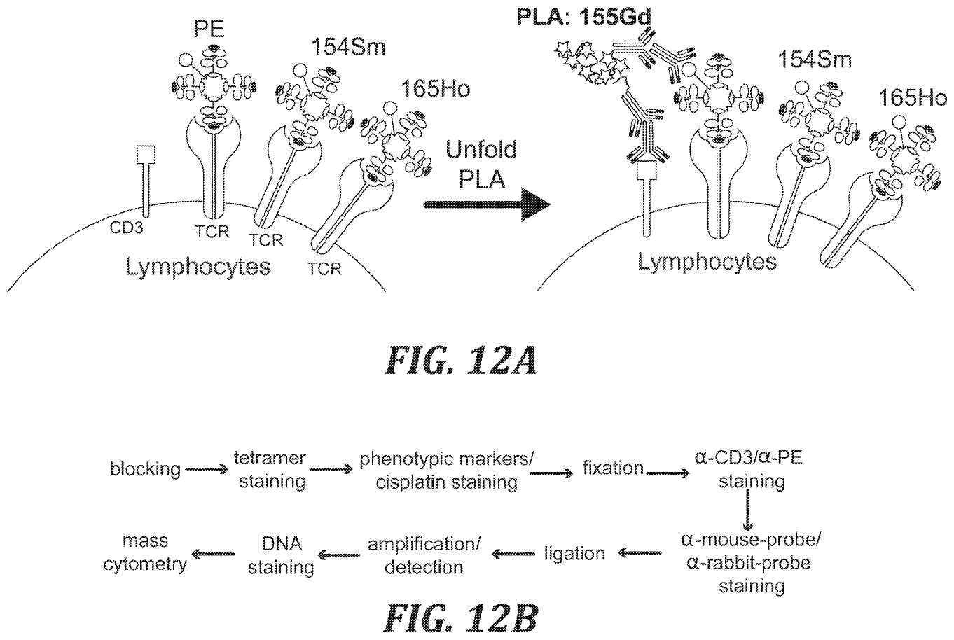

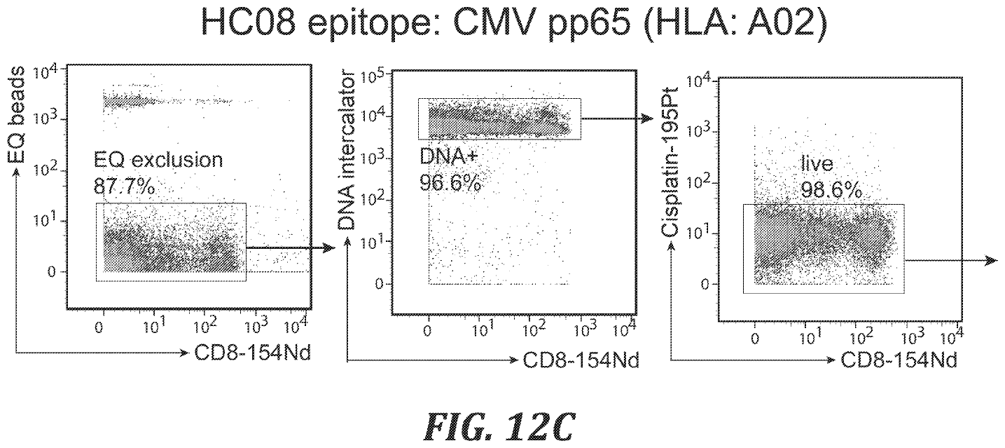

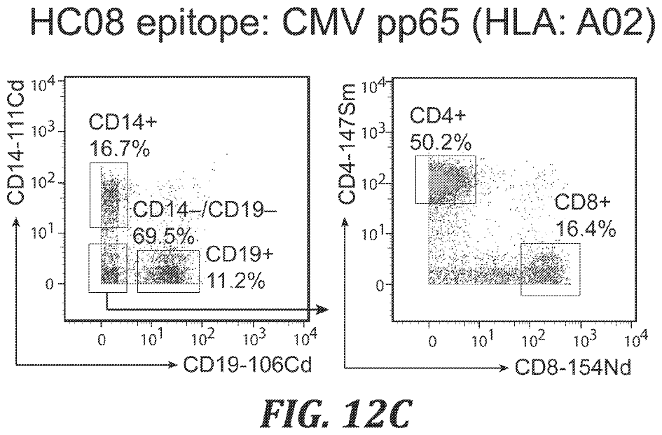

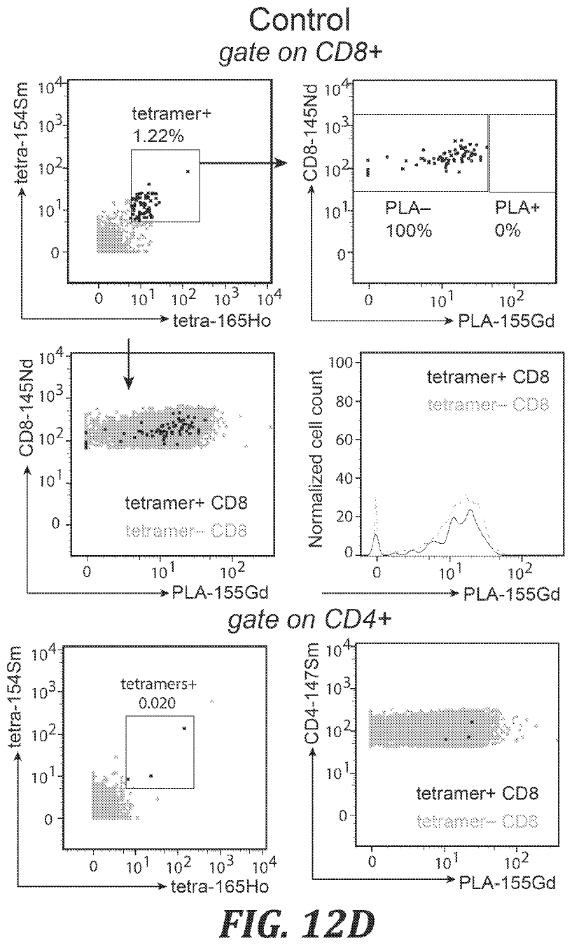

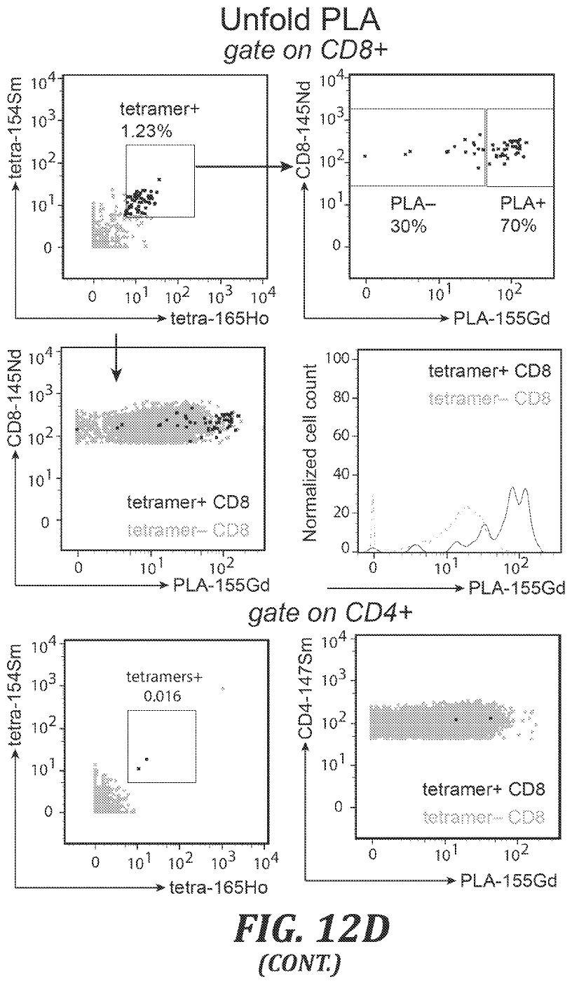

In the following experiment, Unfold PLA was tested with a tetramer barcoding strategy for the detection of virus-specific T cells on mass cytometry. Experimental design and protocol were introduced in FIGS. 12A and 12B. PBMCs from HC08 were treated with a cocktail of tetramer-PE, tetramer-154Sm and tetramer-165Ho bearing an HLA-A*02:01-restricted peptide antigen from CMV pp65. After being stained with phenotypic markers including CD4, CD8, CD14, CD19 and so on, cells were fixed and treated with mouse anti-CD3 and rabbit anti-PE. The cells were then treated with secondary antibodies anti-mouse IgG-probe and anti-rabbit IgG-probe. Unfold PLA was then performed, and the detection oligo linked with 155Gd was used for detection on mass cytometry. As shown in FIGS. 12C and 12D, 70% of double tetramer (154Sm and 165Ho) positive CD8 T cells also had a PLA signal, suggesting they were true CMV pp65-specific T cells. The remaining double tetramer positive T cells (i.e., 30%) were likely false positive hits.

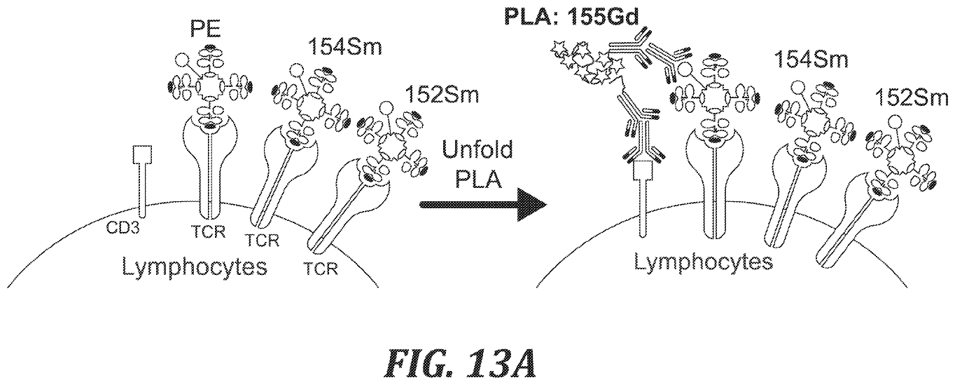

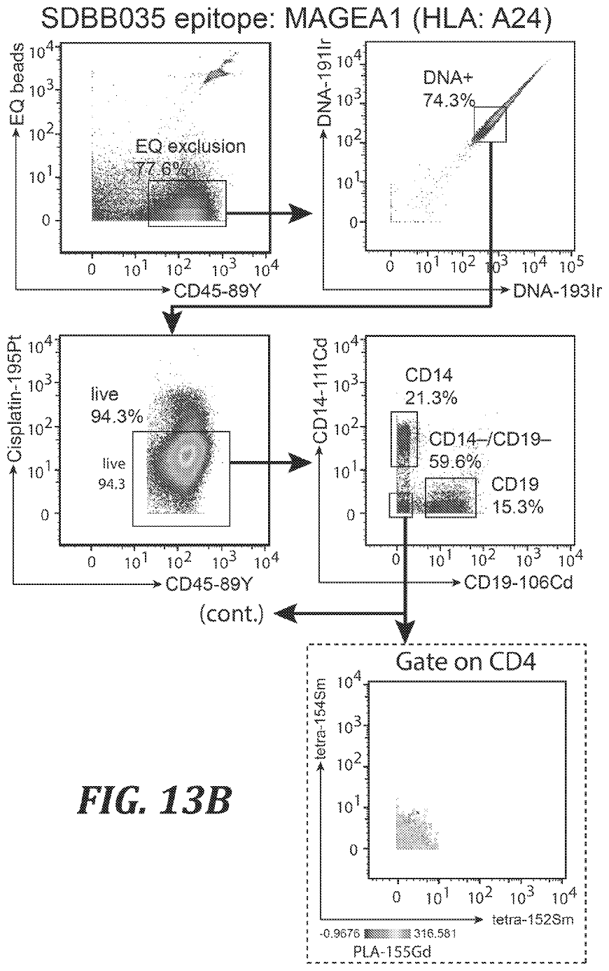

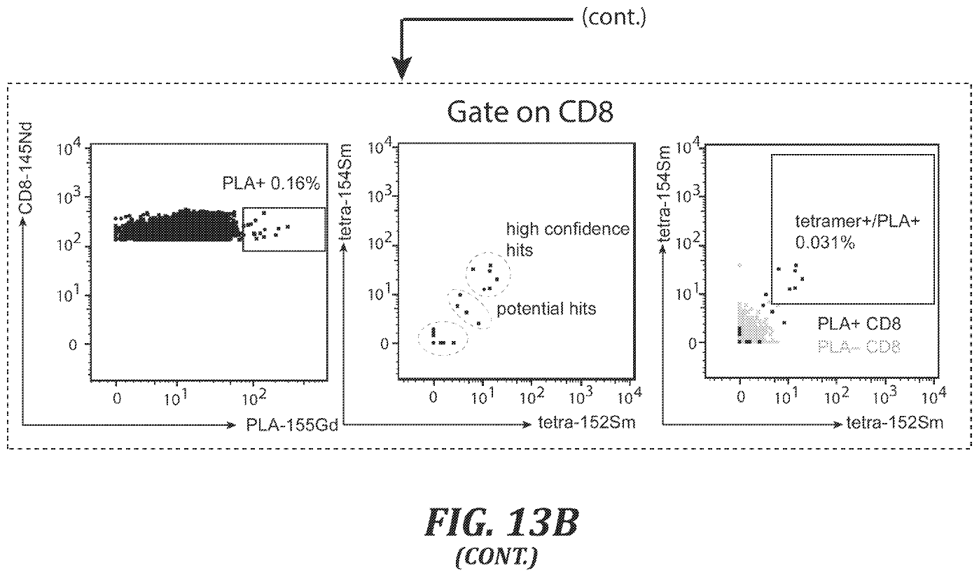

After confirming that Unfold PLA works on mass cytometry, a more challenging model using tumor-specific antigen (MAGE-A1 HLA-A*24 restricted) was tested (FIG. 13). 0.031% of total CD8 T cells had a high double tetramer (152Sm and 154Sm) signal with positive PLA signal, suggesting these cells were high confidence MAGE-A1-specific T cells. It worth noting that some CD8 T cells had weak tetramer staining with a high PLA signal, suggesting these CD8 T cells were also likely to be antigen-specific T cells. There were also some CD8 T cells with weak tetramer staining and no PLA signal, suggesting these CD8 T cells are not true antigen-specific T cells. This is a good example to demonstrate that PLA can help to identify true but low affinity antigen-specific T cells that often show weak tetramer staining.

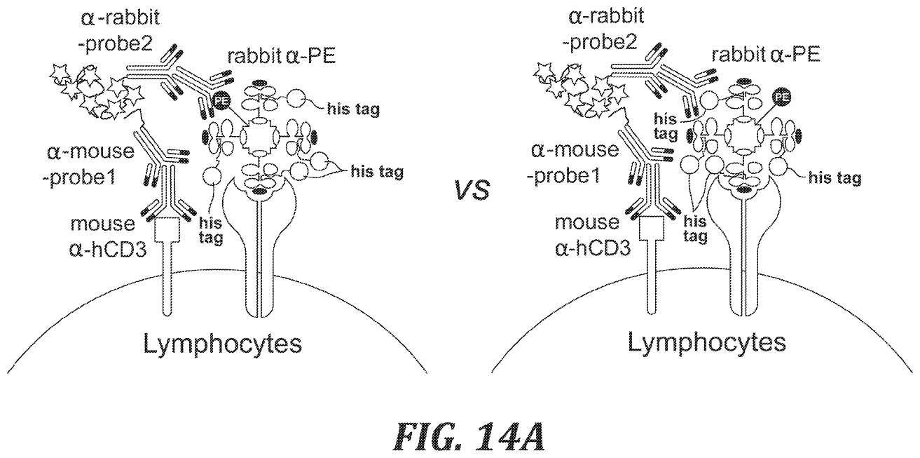

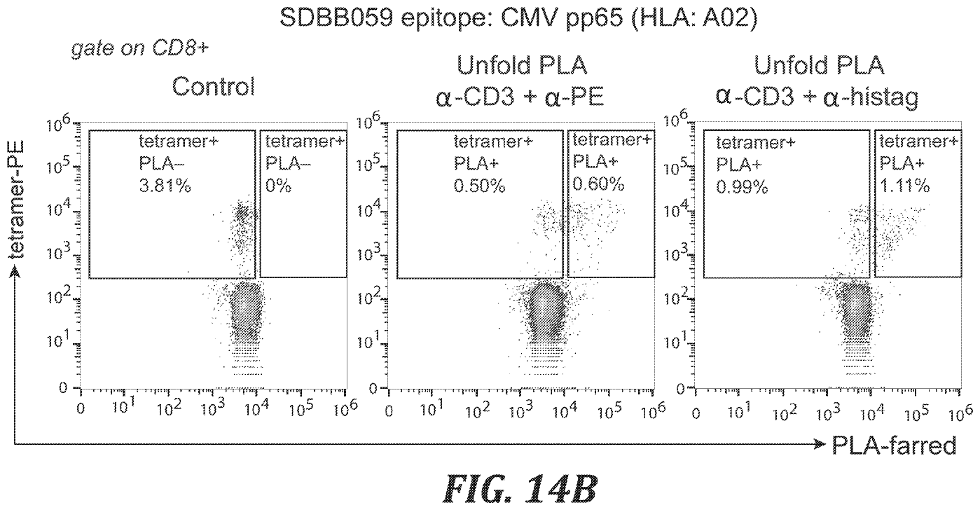

To integrate PLA into the current triple tetramer coding strategy, an alternative primary antibody combination of mouse anti-hCD3 and rabbit anti-his tag was tested and compared with the combination of mouse anti-hCD3 and rabbit anti-PE (FIG. 14A). First, the tetramer was prepared using streptavidin-PE and MHC-I monomers bearing a his-tag. PBMCs from SDBB059 were stained with the tetramer bearing the antigen CMV pp65 and Unfold PLA was performed. In this design, PLA signal could be generated using two primary antibody combinations: anti-CD3/anti-PE (FIG. 14A, left picture) and anti-CD3/anti-his-tag (FIG. 14A, right picture). The flow cytometric analysis indicated that the PLA signal generated by anti-CD3/anti-his-tag was comparable with that of anti-CD3/anti-PE (FIG. 14B).

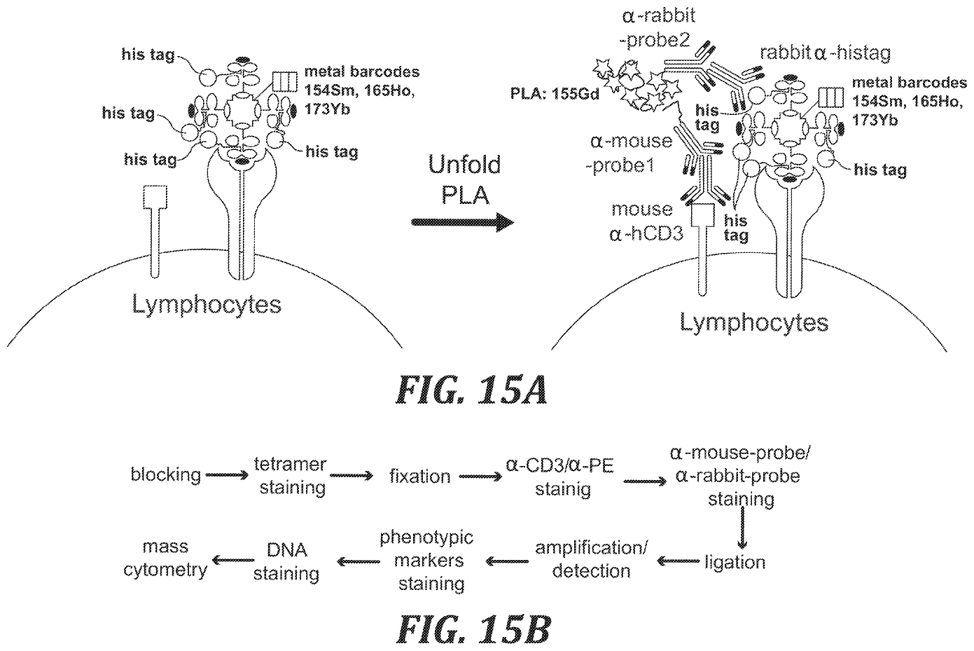

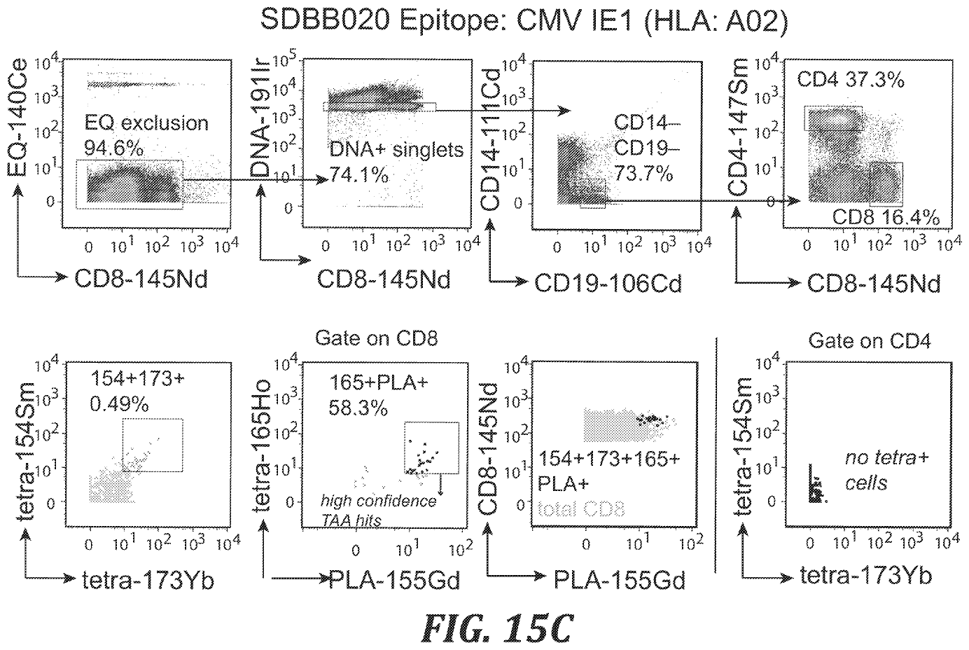

Then Unfold PLA was performed with his-tag-tetramer triple-barcoding. PBMCs from SDBB020 were stained with a cocktail of tetramer-154Sm, 165Ho and 173Yb bearing antigen CMV IE1 (FIG. 15A). These tetramers were prepared with the MHC-I monomers with his-tag. As in the previous experiment, 155Gd was used for the PLA signal. As shown in FIG. 15C, around half of tetramer-154Sm and tetramer-173Yb positive CD8 T cells (0.49% of total CD8) had tetramer-165Ho and PLA-155Gd signal, suggesting the percentage of true CMV IE1-specific CD8 was ˜0.29% of total CD8. In contrast, CD4 population showed low background in all tetramer and PLA channels.

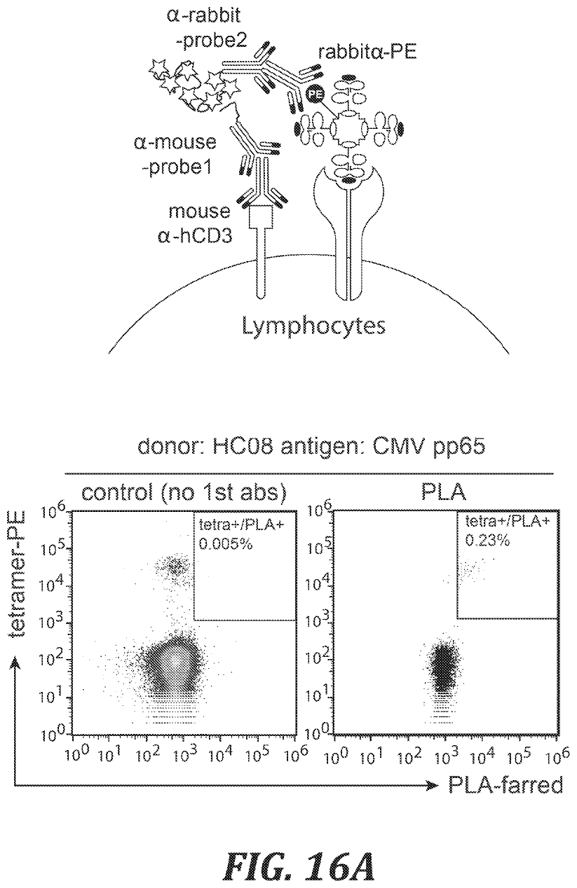

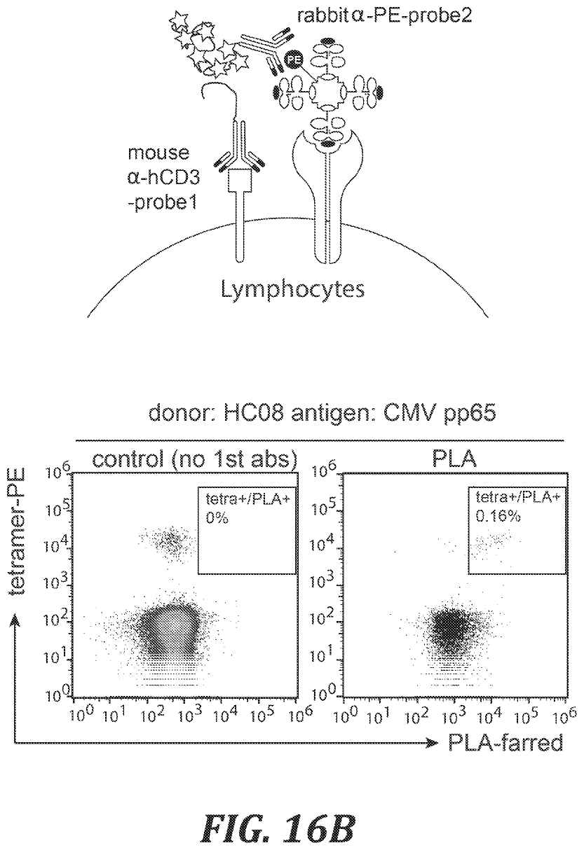

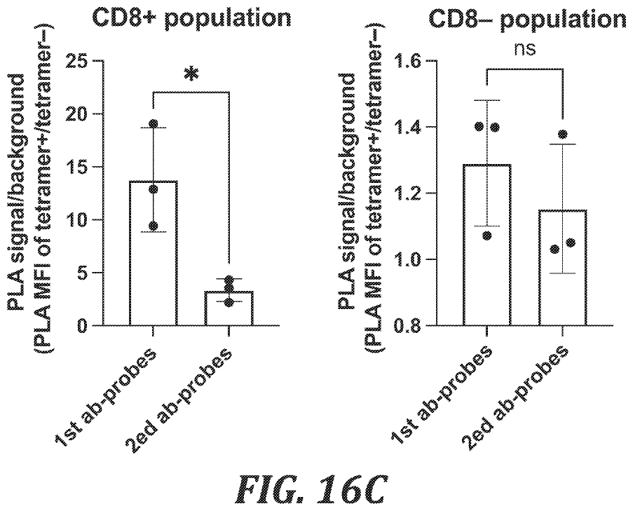

To further optimize the PLA protocol for stronger signaling, PLA oligos were directly conjugated to primary antibodies (anti-CD3 and anti-PE) using the probe conjugation kit (Navinci) as shown in FIG. 16. PBMCs from donor HC08 were stained with the tetramer bearing CMV pp65 peptide, and then treated with anti-CD3-probe1 and anti-PE-probe2 for generating PLA signal. In parallel, PLA using the original protocol (anti-CD3/anti-PE staining followed by secondary antibody-probes staining) was performed for comparison. Flow cytometric analysis indicates that the PLA using primary antibody-probes also works well and can show stronger signal/noise ratio.

### Methods

Performing PLA Targeting CD3 and p:MHCI Tetramer Using Human PBMCs with Anti-hCD3 and Anti-PE for Flow Cytometric Analysis (Unfold flowPLA Kit).

PBMCs from donor SDBB037 or SDBB014 (3 M cells per well) were seeded in a 96-well plate. After treating with blocking buffer for 30 min at 37° C., cells were treated with corresponding p:MHCI tetramer-PE (dilution factor 1:25) for 30 min at room temperature, followed by fixation with 2% paraformaldehyde (PFA) for 30 min at room temperature or overnight at 4° C. Then cells were incubated with primary antibodies anti-hCD3 (working conc. 2.5 mg/mL) and rabbit anti-PE (0.2 mg/mL). Subsequently, cells were treated with secondary antibody (anti-rabbit and anti-mouse)-probes provided in the PLA kit. Subsequently the cells were subjected to ligation, amplification, and detection steps according to Unfold PLA kit user manual. And finally, cells were treated with anti-hCD8-pacific blue before flow cytometric analysis. In the PLA experiment using Smart-tubes, the experimental procedures are similar except using Smart-tube fixation according to user's manual instead of 2% PFA fixation.

Comparison of the Sensitivity and Specificity of Tetramer Staining and PLA Using MAGE-A1 and MAGE-A4-Specific TCR-T Cells (Unfold flowPLA Kit).

MAGE-A1 and MAGE-A4-specific TCR-T cells were prepared in house. On the day of experiment, TCR-T cells were pre-stained with carboxyfluorescein succinimidyl ester (CFSE) (5 mM in PBS) on ice for 30 min. Then TCR-T cells were mixed with PBMCs from SDBB020 as a ratio of 0.05% in total cells. After being stained with MAGE-A1 or A4 tetramer-PE (1:25), cell mixture was subjected to Unfold flowPLA procedures and analyzed by flow cytometry.

### General Procedure for Tetramer Cocktail Preparation for CyTOF PLA.

### Version 1. Tetramer Cocktails Containing a PE Tag

Peptide (working conc. 50 mM) was added in corresponding HLA monomers (working conc. 100 mg/mL) and the mixture was incubated at 4° C. overnight. Then a mixture of streptavidin conjugates (metal1, metal2 and PE) was add to the exchanged monomer (7.5 mL streptavidin mixture for 50 mL exchanged monomer) and incubated at room temperature for 10 min. Biotin (working conc. 10 mM) was added to quench the reaction at room temperature for 10 min. Then the tetramer cocktail was ready to use.

### Version 2. Tetramer Cocktails Prepared Using His Tag Monomers

Peptide (working conc. 50 mM) was added in corresponding his tag HLA monomers (working conc. 100 mg/mL) and the mixture was incubated at 4° C. overnight. Then a mixture of streptavidin conjugates (metal1, metal2 and metal3) was add to the exchanged monomer (7.5 mL streptavidin mixture for 50 mL exchanged monomer) and incubated at room temperature for 10 min. Biotin (working conc. 10 mM) was added to quench the reaction at room temperature for 10 min. Then the tetramer cocktail was ready to use.

Performing PLA Targeting CD3 and p:MHCI Tetramer Using Human PBMCs with Anti-hCD3 and Anti-PE for Mass Cytometry Analysis (Unfold PLA Kit for Mass Cytometry).

PBMCs were seeded in a 96-well plate (3 M cells per well). After treating with blocking buffer for 30 min at 37° C., cells were treated with corresponding p:MHCI tetramer cocktail (version 1) for 30 min at room temperature. Subsequently, cells were treated with surface antibody-metals, cisplatin and followed by fixation with 2% paraformaldehyde (PFA) for 30 min at room temperature or overnight at 4° C. Then cells were incubated with primary antibodies anti-hCD3 (working conc. 2.5 mg/mL) and rabbit anti-PE (0.2 mg/mL) at room temperature for 30 min. Subsequently, cells were treated with secondary antibody (anti-rabbit and anti-mouse)-probes provided in the PLA kit. Subsequently the cells were subjected to ligation and amplification steps according to Unfold PLA user manual. Then cells were treated with detection oligo-Gd155 (1:200) provided by Navinci for 15 min at 37° C. After being washed with the CyTOF washing buffer (Navinci), cells were stained with DNA intercalator (191Ir/193Ir) and then submitted for mass cytometric analysis.

Performing PLA Targeting CD3 and p:MHCI Tetramer Using Human PBMCs with Anti-hCD3 and Anti-His Tag for Mass Cytometry Analysis (Unfold PLA Kit for Mass Cytometry).

PBMCs were seeded in a 96-well plate (3 M cells per well). After treating with blocking buffer for 30 min at 37° C., cells were treated with corresponding p:MHCI tetramer cocktail (version 2) for 30 min at room temperature. Then cells were stained with cisplatin and followed by fixation with 2% paraformaldehyde (PFA) for 30 min at room temperature or overnight at 4° C. Then cells were incubated with primary antibodies anti-hCD3 (working conc. 2.5 mg/mL) and rabbit anti-his tag (2.5 mg/mL). Subsequently, cells were incubated with secondary antibody (anti-rabbit and anti-mouse)-probes provided in the PLA kit. Subsequently the cells were subjected to ligation and amplification steps according to Unfold PLA user manual. Then cells were treated with detection oligo-Gd155 (1:200) provided by Navinci for 15 min at 37° C. After being washed with the CyTOF washing buffer (Navinci), cells were stained with surface antibody-metals, followed by being stained with DNA intercalator (191Ir/193Ir) and then submitted for mass cytometric analysis.

Performing PLA Targeting CD3 and p:MHCI Tetramer Using Human PBMCs with Anti-hCD3-Probe1 and Anti-PE-Probe2 for Flow Cytometry Analysis.

PLA oligo probe1 and PLA oligo probe 2 were provided in the probe conjugation kit from Navinci. The conjugation reactions were performed according to user's manual.

PBMCs (3 M cells per well) were seeded in a 96-well plate. After treating with blocking buffer for 30 min at 37° C., cells were treated with corresponding p:MHCI tetramer-PE (dilution factor 1:25) for 30 min at room temperature, followed by fixation with 2% paraformaldehyde (PFA) for 30 min at room temperature or overnight at 4° C. Then cells were incubated with primary antibodies-probes: anti-hCD3-probe1 (working conc. 2.5 mg/mL) and rabbit anti-PE-probe2 (0.2 mg/mL) at room temperature for 30 min. Subsequently the cells were subjected to ligation, amplification, and detection steps according to Unfold PLA kit user manual. And finally, cells were treated with anti-hCD8-pacific blue before flow cytometric analysis.

### Example 4

### Unfold PLA Assay for Antigen-Specific B Cells

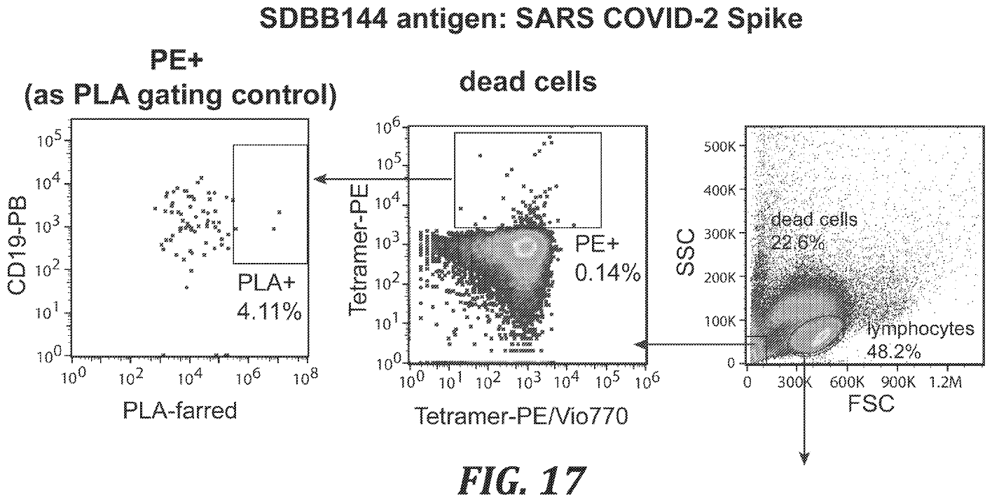

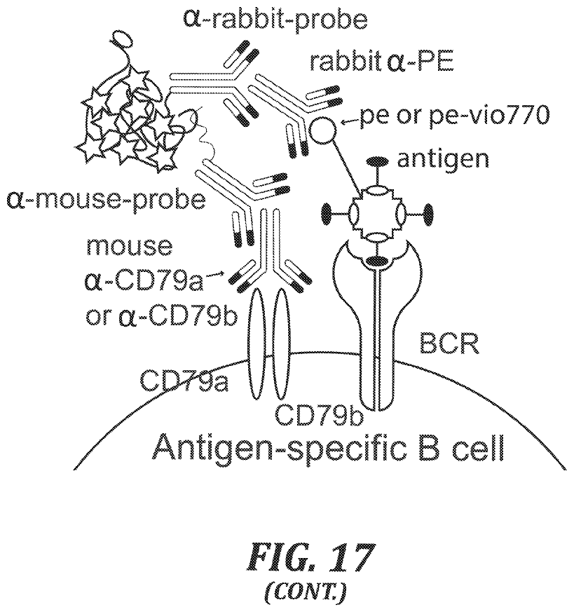

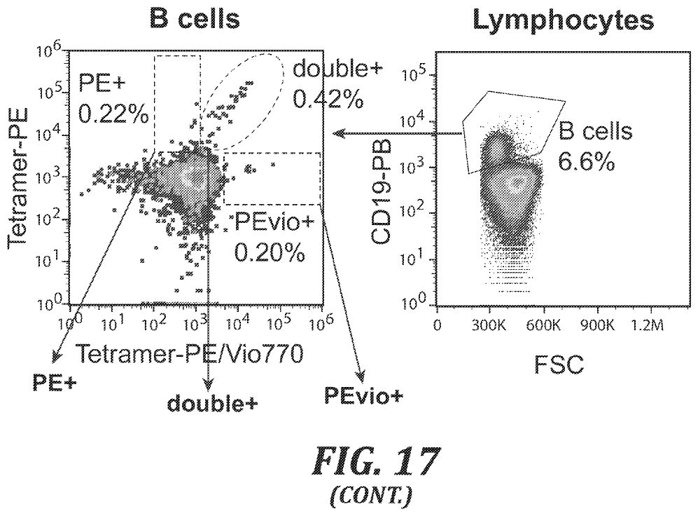

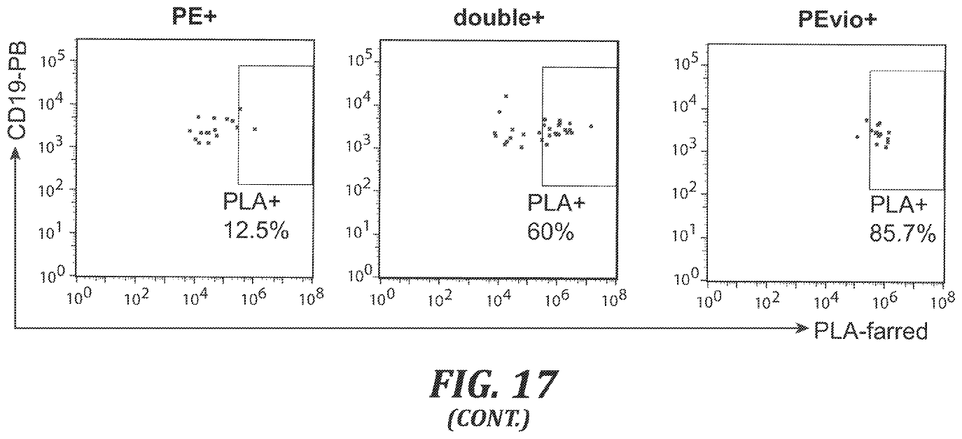

Beyond detecting antigen-specific T cells, PLA could also be used to improve the specificity of antigen-specific B cells. Currently, the general high background of tetramer staining on B cells is considered one of the major challenges for the identification for antigen-specific B cells. As shown in FIG. 17, the PLA signal between BCR and its co-receptors CD79a/b would be an unbiased indication for antigen specificity. In a pilot experiment, PBMCs from SDBB144 were first stained with a cocktail of PE-labelled and PE/vio770-labelled SARS COVID-2 spike antigen complexes. Unfold PLA were performed with the primary antibody combination of mouse anti-79a/b and rabbit anti-PE antibodies. Results showed that only 60% of double PE+ and PE/vio770+ B cells were PLA+. In contrast, PE+ and PE/vio770− B cells contain 12.5% PLA+ cells while PE- and PE/vio770+ B cells contain 85.7% PLA+ cells. These observations suggest that detection of antigen-specific B cells relying on tetramer staining has low specificity and PLA could help to improve the accuracy.

### Methods

Performing PLA Targeting CD79a/b and SARS-CoV-2 Spike Antigen Complexes to Identify Antigen-Specific B Cells from Human PBMCs (Unfold PLA Kit for Mass Cytometry).

SARS-COV-2 spike antigen complexes bearing fluorophore PE or PE/Vio770 were prepared according to user manual (Miltenyi, 130-128-032) and the complexes bearing two different fluorophores were mixed as a cocktail for downstream staining.

PBMCs from donor SDBB144 (3 M cells per well) were seeded in a 96-well plate. After treating with blocking buffer for 30 min at 37° C., cells were treated with the cocktail of SARS-COV-2 spike antigen complexes-PE and PE/vio770 for 30 min at room temperature, followed by been stained with anti-hCD19-pacific blue at room temperature for 30 min. Then cells were fixed with 2% paraformaldehyde (PFA) for 30 min at room temperature or overnight at 4° C. Then cells were incubated with primary antibodies anti-hCD3 (working conc. 2.5 mg/mL) and rabbit anti-PE (0.2 mg/mL) at room temperature. Subsequently, cells were treated with secondary antibody (anti-rabbit and anti-mouse)-probes provided in the PLA kit. Subsequently the cells were subjected to ligation, amplification, and detection steps according to Unfold PLA kit user manual. And finally, cells were analyzed by flow cytometry.

### Example 5

### PLA Assay for TCR Discovery

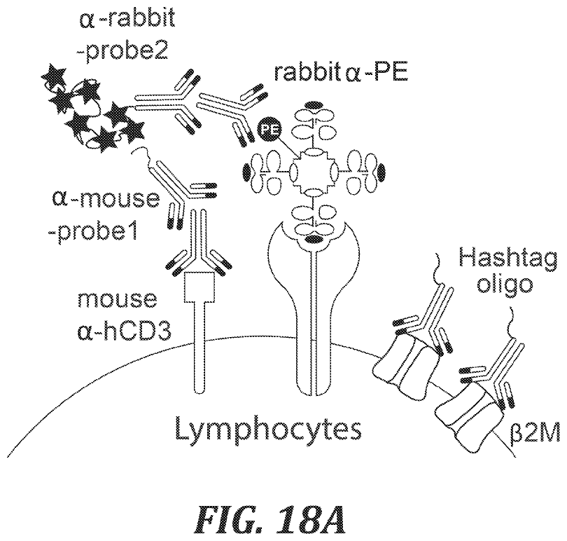

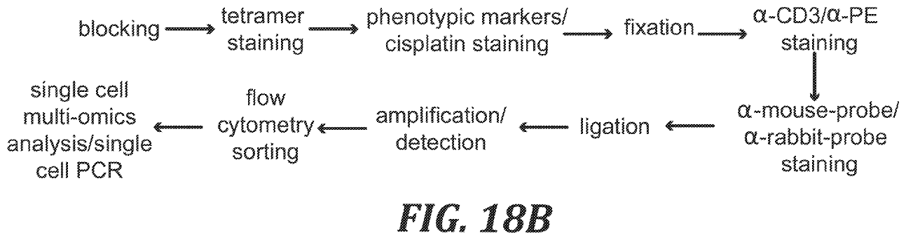

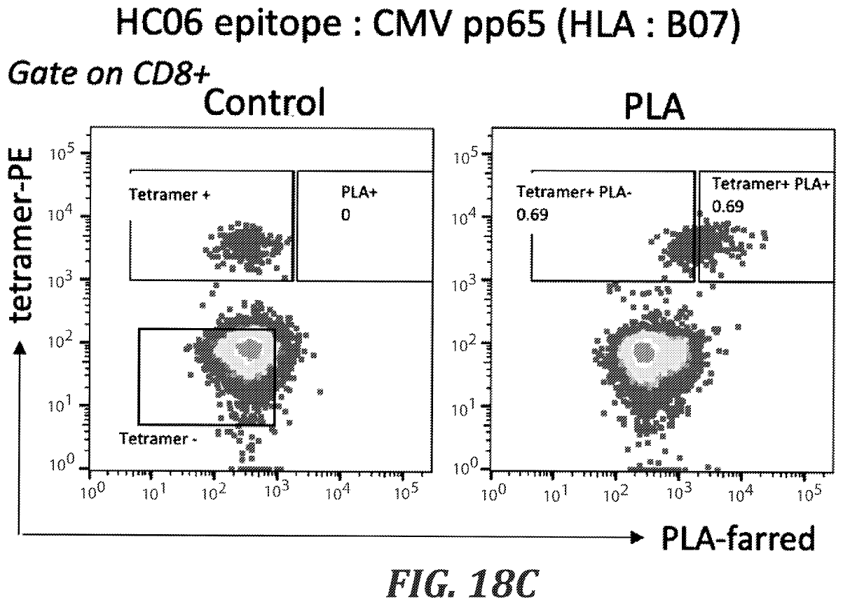

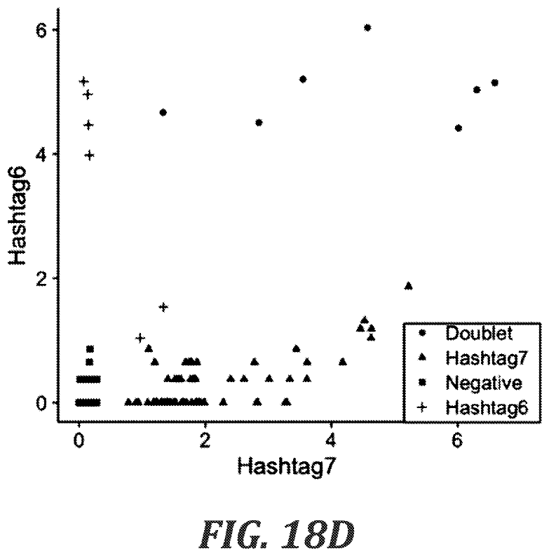

PLA can also be integrated in antigen-specific TCR discovery workflows to derive relevant TCR sequences using single cell sequencing methods. To this end, oligo conjugated hashtag antibodies were combined with unfold PLA. The combination of antibodies is shown in FIG. 18A and the protocol is shown in FIG. 18B. After being stained with tetramer bearing PE, cells were stained with phenotypic markers and oligo hashtags (hashtag6 or hashtag7). Two experiments were run in parallel. All the cells in experiment one were stained with hashtag6 and all the cells in experiment two were stained with hashtag7. After fixation, all cells from experiment one were stained with mouse anti-human CD3 and rabbit anti-PE antibodies, followed by secondary antibody-probes targeting the corresponding primary antibodies. After ligation and amplification, detection oligo bearing a far-red dye Atten647 was used to generate the PLA signal in experiment one. For single cell sequencing, the Tetramer+ PLA+ cells carrying hashtag6 from experiment one and Tetramer− cells carrying hashtag7 from experiment two were sorted (FIG. 18C), mixed and loaded to 10× genomics K chip for single cell encapsulation. The library preparations for feature barcode and VDJ were performed subsequentially and sequenced using next-generation sequencing (NGS). The sequencing results of VDJ library were analysed with Cellranger for TCR alignment, VDJ annotation and read counting for each cell. The feature barcode library was analysed in parallel for counting the hashtag oligo sequences of each cell. As shown in FIG. 18D, the hashtag6 labelled Tetramer+ PLA+ cells and hashtag7 labelled Tetramer− cells were distinguished by normalized hashtag intensities. Cell doublets and negative cells were abandoned in subsequential TCR analysis. In total, 8 unique clonotypes were identified from Tetramer+ PLA+(43 clonotypes were identified from Tetramer− cells). The TCR sequences of Tetramer+ PLA+ cells (hashtag6) are listed in Table 1. Those clonotypes were searched in our internal database for matching sequences specific for the same epitope (CMV pp65) and same HLA type (HLA-B*07:02). One of the eight clonotypes was found to have a CDR3 amino acid sequence identical to a CMV pp65-specific TCR in from a reference sample. This result indicates that the CMV pp65 specific CD8+ T cells could be identified using PLA signal converted in oligo hashtags. Thus, the PLA method could be applied to multiplexed single cell TCR sequencing for antigen-specific TCR discovery. Relevant TCR discovered by this method could be then developed for therapeutics applications.

The TCRs of antigen specific PLA+ cells were also sequenced with single cell PCR (scPCR). Tetramer+ PLA+ cells were sorted as gated in FIG. 18C. Targeted PCR of TCR alpha chain and beta chain were performed for each sorted cell and the PCR products were sequenced with sanger sequencing. The sequencing results were blasted using IgBlast for the alignment to CDR3 regions and annotation of VDJ names. Table 2 lists the TCR sequences obtained from two cells. TRAV8-1 and TRBV24-1 were paired from the same cell. TRBV4-3 were from another cell and the sequence has been found to have a CDR3 amino acid sequence identical to a prior discovered CMV pp65-specific, HLA-B*07:02-restricted TCR sequence from a different healthy donor. This result further demonstrates that PLA, followed by either single cell PCR or single cell sequencing, can be applied for discovery of antigen specific TCR sequences.

TABLE 1
TCR sequences of Tetramer + PLA + cells from single cell sequencing

| | | | | Detected in |
|---|---|---|---|---|
| | | | | reference |
| V.name | J.name | CDR3.nt | CDR3.aa | sample |
| TRBV4-1 | TRBJ2-7 | TGCGCCAGCAGCCAAGA | CASSQETGLYEQ | No |
| | | AACAGGGCTCTACGAGCA | YF | |
| | | GTACTTC | (SEQ ID NO: 4) | |
| | | (SEQ ID NO: 3) | | |
| TRBV4-3 | TRBJ1-1 | TGCGCCAGCAGCCCCCAG | CASSPQRNTEAFF | Yes |
| | | AGAAACACTGAAGCTTTC | (SEQ ID NO: 6) | |
| | | TTT | | |
| | | (SEQ ID NO: 5) | | |
| TRBV28 | TRBJ2-3 | TGTGCCAGCAGCCCCGAC | CASSPDEGTGGN | No |
| | | GAAGGGACAGGGGGCAA | NADTQYF | |
| | | CAACGCAGATACGCAGTA | (SEQ ID NO: 8) | |
| | | TTTT | | |
| | | (SEQ ID NO: 7) | | |
| TRBV2 | TRBJ2-7 | TGTGCCAGCAGTGAACTA | CASSELAGAYEQ | No |
| | | GCGGGGGCCTACGAGCA | YF | |
| | | GTACTTC | (SEQ ID NO: 10) | |
| | | (SEQ ID NO: 9) | | |
| TRBV5-1 | TRBJ2-5 | TGCGCCAGCAGCTTGGCC | CASSLASTGPQET | No |
| | | TCGACAGGACCTCAAGAG | QYF | |
| | | ACCCAGTACTTC | (SEQ ID NO: 12) | |
| | | (SEQ ID NO: 11) | | |
| TRBV7-3 | TRBJ2-6 | TGTGCCAGCAGCTTAATT | CASSLIGFSGANV | No |
| | | GGGTTCTCTGGGGCCAAC | LTF | |
| | | GTCCTGACTTTC | (SEQ ID NO: 14) | |
| | | (SEQ ID NO: 13) | | |
| TRAV9-2 | TRAJ45 | TGTGCTCTGTGGTATTCA | CALWYSGGGAD | No |
| | | GGAGGAGGTGCTGACGG | GLTF | |
| | | ACTCACCTTT | (SEQ ID NO: 16) | |
| | | (SEQ ID NO: 15) | | |

TABLE 2
TCR sequences of Tetramer + PLA + cells from single cell PCR

| | | | | Detected in |
|---|---|---|---|---|
| | | | | reference |
| V.name | J.name | CDR3.nt | CDR3.aa | sample |
| TRAV8-1 | TRAJ34 | GGTGCCCGTGAAAGAAC | GARERTPIKAH | No |
| | | ACCGATCAAGGCTCATC | (SEQ ID NO: 18) | |
| | | (SEQ ID NO: 17) | | |
| TRBV24-1 | TRBJ2-2 | GCCACCAGTGATTGGGA | ATSDWDDSTGEL | No |
| | | CGACAGCACCGGGGAGC | F | |
| | | TGTTT | (SEQ ID NO: 20) | |
| | | (SEQ ID NO: 19) | | |
| TRBV4-3 | TRBJ1-1 | GCCAGCAGCCCCGCCCG | ASSPARNTEAF | Yes |
| | | GAACACTGAAGCTTTC | (SEQ ID NO: 22) | |
| | | (SEQ ID NO: 21) | | |

### Methods

Performing PLA Targeting CD3 and p:MHCI Tetramer Using Human PBMCs with Anti-hCD3 and Anti-PE for Single Cell Sequencing Analysis (Unfold PLA Kit for Flow Cytometry Incorporation with Either 10× Genomics or Single Cell PCR).

PBMCs from donor HC06 (20 M cells per tube) were seeded in 1.5 ml Eppendorf tubes. After treating with blocking buffer for 30 min at 37° C., cells were treated with corresponding p:MHCI tetramer-PE (dilution factor 1:25) for 30 min at room temperature. For 10× Genomics workflow, cells were split into two experiments and further stained with anti-hCD8-BV510, anti-hCD8a (Biolegend TotalSeq™-C0080) as well as either anti-human hashtag6 (Biolegend TotalSeq™-C0256) antibody for experiment one or anti-human hashtag7 (Biolegend TotalSeq™-C0257) antibody for experiment two. For single cell PCR, cells were further stained with anti-hCD8-BV510. For PLA, all cells were fixed with 0.25 mg/ml DSP (200 μl/1M cell) at room temperature for 30 minutes followed by quenching with 20 mM TrisHCL (pH7.0) at room temperature for 10 minutes and kept overnight. The cells were then incubated with primary antibodies anti-hCD3 (working conc. 2.5 mg/mL) and rabbit anti-PE (0.2 mg/mL). Subsequently, cells were stained with secondary antibody (anti-rabbit and anti-mouse)-probes provided in the Unfold PLA kit. Subsequently, the cells were subjected to ligation, amplification, and detection steps according to the procedures from the Unfold PLA kit. Then cells were acquired and sorted by flow cytometry. For 10×, Tetramer+ PLA+(hashtag6) from experiment one were sorted and combined with Tetramer− PLA− (hashtag7) sorted from experiment two and loaded onto a 10× Chip K for single cell encapsulation. Then sequencing libraries for gene expression, VDJ and feature barcode were prepared from encapsulated cells according to the manufacturer's recommendations (10× Genomics, Next GEM 5′ V2) and sequenced (Illumina, NovaSeq6000). For single cell PCR, PLA+ cells were sorted into a 96-well plate, one cell per well. Reverse transcription of single cell transcriptome was performed in the single wells and the product from each well was split into two wells for alpha chain and beta chain amplification using pre-designed primer panels. The PCR product was sequenced using Sanger sequencing and mapped to reference TCR with IgBlast.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for detecting a target cell in a sample, comprising:
contacting the target cell with a cell selector component for which the target cell is specific, the cell selector component being bound to a first oligonucleotide;
contacting the target cell with a targeting component specific for a receptor on the target cell, the targeting component being bound to a second oligonucleotide; and
forming a nucleic acid template by ligating the first oligonucleotide and the second oligonucleotide.

2. The method of claim 1, wherein the first oligonucleotide is directly bound to the cell selector component.

3. The method of claim 1 or 2, wherein the second oligonucleotide is directly bound to the targeting component.

4. The method of claim 1 or 2, wherein the first oligonucleotide is bound to a first secondary antibody that is bound to the cell selector component.

5. The method of claim 4, wherein the first secondary antibody is bound to a myc-tag, a his-tag, or a fluorescent tag on the cell selector component.

6. The method of claim 3, wherein the second oligonucleotide is bound to a second secondary antibody that is bound to the targeting component.

7. The method of any one of claims 1-6, wherein the cell selector component comprises a peptide-MHC multimer.

8. The method of claim 7, wherein the peptide-MHC multimer comprises a peptide-MHC tetramer

9. The method of any one of claims 1-7, wherein the peptide-MHC multimer comprises streptavidin or dextran.

10. The method of claim 9, wherein the first oligonucleotide is bound to streptavidin or dextran.

11. The method of any one of claims 1-6, wherein the cell selector component comprises a B cell specific antigen.

12. The method of any one of claims 1-11, wherein the targeting component comprises an antibody or antigen binding fragment thereof, a fusion protein, an aptamer, or a combination thereof.

13. The method of any one of claims 1-10 or 12, wherein the receptor is CD3 or the T cell receptor (TCR).

14. The method of any one of claims 1-10, 12, or 13 wherein the target cell is a T cell.

15. The method of any one of claims 1-6 or 11, wherein the receptor is CD79 of the B cell receptor (BCR).

16. The method of any one of claims 1-6, 11, or 15 wherein the target cell is a B cell.

17. The method of any one of claims 1-16, wherein the cell selector component is no more than 50 nanometers (nm) from the targeting component.

18. The method of any one of claims 1-17, wherein the cell selector component is no more than 40 nm from the targeting component.

19. The method of any one of claims 1-18, wherein the cell selector component is no more than 35 nm from the targeting component.

20. The method of any one of claims 1-19, wherein the cell selector component is no more than 30 nm from the targeting component.

21. The method of any one of claims 1-20, wherein the cell selector component is no more than 25 nm from the targeting component.

22. The method of any one of claims 1-21, further comprising contacting the first and second oligonucleotides with a connector oligonucleotide.

23. The method of any one of claims 1-22, further comprising amplifying the nucleic acid template.

24. The method of claim 23, wherein the amplifying the nucleic acid template comprises rolling circle amplification.

25. The method of claim 23 or 24, wherein the amplifying the nucleic acid template comprises producing an amplification product.

26. The method of claim 25, further comprising detecting the amplification product.

27. The method of claim 26, wherein the detecting the amplification product comprises forming a labeled amplification product by contacting the amplification product with a probe comprising a fourth oligonucleotide and a label, the fourth oligonucleotide having a sequence that is substantially complementary to at least a portion of the amplification product.

28. The method of claim 27, wherein the label is a fluorescent label.

29. The method of claim 27, wherein the label is a heavy metal tag.

30. The method of claim 27, wherein the label is an oligonucleotide tag or a DNA barcode.

31. The method of claim 27 or 28, wherein the detecting the amplification product comprises imaging the labeled amplification product using flow cytometry.

32. The method of claim 27 or 29, wherein the detecting the amplification product comprises using time-of-flight mass spectrometry.

33. The method of claim 27 or 30, wherein the detecting the amplification product comprises using sequencing or PCR.

34. The method of any one of claims 1-33, wherein the ligating the first oligonucleotide and the second oligonucleotide comprises using a protein ligase.

35. The method of any one of claims 1-33, wherein the ligating the first oligonucleotide and the second oligonucleotide comprises using a chemical ligase.

36. The method of any one of claims 1-33, wherein the ligating the first oligonucleotide and the second oligonucleotide comprises using a nucleic acid ligase.

37. The method of any one of claims 1-36, wherein the sample is a tissue sample or a cell sample.

38. The method of any one of claims 1-37, further comprising sequencing the target cell receptor.

39. A kit for detecting a target cell in a sample comprising:
a cell selector component for which the target cell is specific, the cell selector component being bound to a first oligonucleotide;
a targeting component specific for a second antigen on the target cell, the targeting component being bound to a second oligonucleotide;
a ligation reagent; and
written instructions for using the cell selector component and the targeting component to detect the target cell.

40. The kit of claim 39, further comprising a connector oligonucleotide.

41. The kit of claim 39 or 40, further comprising a probe comprising a fourth oligonucleotide and a label.

42. The kit of claim 41, wherein the label is a fluorescent label.

43. The kit of claim 41, wherein the label is a heavy metal tag.

44. The kit of claim 41, wherein the label is an oligonucleotide tag or a DNA barcode.

45. The kit of any one of claims 39-44, wherein the ligation reagent comprises a protein ligase.

46. The kit of any one of claims 39-44, wherein the ligation reagent comprises a chemical ligase.

47. The kit of any one of claims 39-44, wherein the ligation reagent comprises a nucleic acid ligase.

48. A synthetic construct, comprising:
a cell selector component;
a first oligonucleotide bound to the cell selector component;
a second oligonucleotide bound to the first oligonucleotide; and
a targeting component bound to the second oligonucleotide.

49. The synthetic construct of claim 48, wherein the first oligonucleotide is bound directly to the cell selector component.

50. The synthetic construct of claim 48 or 49, wherein the second oligonucleotide is directly bound to the targeting component.

51. The synthetic construct of claim 48 or 49, wherein the first oligonucleotide is bound to a first secondary antibody that is bound to the cell selector component.

52. The synthetic construct of claim 51, wherein the first secondary antibody is bound to a myc-tag, a his-tag, or a fluorescent tag on the cell selector component.

53. The synthetic construct of claim 48 or 50, wherein the second oligonucleotide is bound to a second secondary antibody that is bound to the targeting component.

54. The synthetic construct of any one of claims 48-53, wherein the cell selector component comprises a peptide-MHC multimer.

55. The synthetic construct of claim 54, wherein the peptide-MHC multimer comprises a peptide-MHC tetramer

56. The synthetic construct of claim 54, wherein the peptide-MHC multimer comprises streptavidin or dextran.

57. The synthetic construct of claim 56, wherein the first oligonucleotide is bound to streptavidin or dextran.

58. The synthetic construct of any one of claims 48-53, wherein the cell selector component comprises a B cell specific antigen.

59. The synthetic construct of any one of claims 48-58, wherein the targeting component comprises an antibody or antigen binding fragment thereof, a fusion protein, an aptamer, or a combination thereof.

60. The synthetic construct of any one of claims 48-57 or 59, wherein the receptor is CD3 or the T cell receptor (TCR).

61. The synthetic construct of any one of claims 48-57, 59 or 60, wherein the target cell is a T cell.

62. The synthetic construct of any one of claims 48-53 or 58, wherein the receptor is CD79 or the B cell receptor (BCR).

63. The synthetic construct of any one of claims 48-53, 58, or 62, wherein the target cell is a B cell.

64. The synthetic construct of any one of claims 48-63, further comprising a connector oligonucleotide bound to the first and second oligonucleotides.


SEQUENCE LISTING (22 sequences)

SEQ ID NO: 1 - protein, 9 aa, synthetic construct
VLEETSVML

SEQ ID NO: 2 - protein, 10 aa, synthetic construct
ELAGIGILTV

SEQ ID NO: 3 - DNA, 42 bp, synthetic construct
  source 1..42: mol_type = other DNA
tgcgccagcagccaagaaacagggctctacgagcagtacttc

SEQ ID NO: 4 - protein, 14 aa, synthetic construct
CASSQETGLYEQYF

SEQ ID NO: 5 - DNA, 39 bp, synthetic construct
  source 1..39: mol_type = other DNA
tgcgccagcagcccccagagaaacactgaagctttcttt

SEQ ID NO: 6 - protein, 13 aa, synthetic construct
CASSPQRNTEAFF

SEQ ID NO: 7 - DNA, 57 bp, synthetic construct
  source 1..57: mol_type = other DNA
tgtgccagcagccccgacgaagggacagggggcaacaacgcagatacgcagtatttt

SEQ ID NO: 8 - protein, 19 aa, synthetic construct
CASSPDEGTGGNNADTQYF

SEQ ID NO: 9 - DNA, 42 bp, synthetic construct
  source 1..42: mol_type = other DNA
tgtgccagcagtgaactagcgggggcctacgagcagtacttc

SEQ ID NO: 10 - protein, 14 aa, synthetic construct
CASSELAGAYEQYF

SEQ ID NO: 11 - DNA, 48 bp, synthetic construct
  source 1..48: mol_type = other DNA
tgcgccagcagcttggcctcgacaggacctcaagagacccagtacttc

SEQ ID NO: 12 - protein, 16 aa, synthetic construct
CASSLASTGPQETQYF

SEQ ID NO: 13 - DNA, 48 bp, synthetic construct
  source 1..48: mol_type = other DNA
tgtgccagcagcttaattgggttctctggggccaacgtcctgactttc

SEQ ID NO: 14 - protein, 16 aa, synthetic construct
CASSLIGFSGANVLTF

SEQ ID NO: 15 - DNA, 45 bp, synthetic construct
  source 1..45: mol_type = other DNA
tgtgctctgtggtattcaggaggaggtgctgacggactcaccttt

SEQ ID NO: 16 - protein, 15 aa, synthetic construct
CALWYSGGGADGLTF

SEQ ID NO: 17 - DNA, 34 bp, synthetic construct
  source 1..34: mol_type = other DNA
ggtgcccgtgaaagaacaccgatcaaggctcatc

SEQ ID NO: 18 - protein, 11 aa, synthetic construct
GARERTPIKAH

SEQ ID NO: 19 - DNA, 39 bp, synthetic construct
  source 1..39: mol_type = other DNA
gccaccagtgattgggacgacagcaccggggagctgttt

SEQ ID NO: 20 - protein, 13 aa, synthetic construct
ATSDWDDSTGELF

SEQ ID NO: 21 - DNA, 33 bp, synthetic construct
  source 1..33: mol_type = other DNA
gccagcagccccgcccggaacactgaagctttc

SEQ ID NO: 22 - protein, 11 aa, synthetic construct
ASSPARNTEAF